(19) United States
(12) Patent Application Publication
(10) Pub. No.: US 2023/0394655 A1
(43) Pub. Date: Dec. 7, 2023

(54) SYSTEM AND METHOD FOR EVALUATING OR PREDICTING A CONDITION OF A FETUS

(71) Applicants: ICHILOV TECH LTD., Tel Aviv (IL); YISSUM RESEARCH DEVELOPMENT COMPANY OF THE HEBREW UNIVERSITY OF JERUSALEM LTD., Jerusalem (IL)

(72) Inventors: Dafna BEN BASHAT, Tel Aviv (IL); Liat BEN SIRA, Tel Aviv (IL); Daphna LINK, Haifa (IL); Netanell AVISDRIS, Tel Aviv (IL); Leo JOSKOWICZ, Jerusalem (IL)

(21) Appl. No.: 18/236,007

(22) Filed: Aug. 21, 2023

Related U.S. Application Data
(63) Continuation of application No. PCT/IL2022/050204, filed on Feb. 21, 2022.
(60) Provisional application No. 63/151,739, filed on Feb. 21, 2021.

(51) Int. Cl. G06T 7/00; G16H 50/20

(52) U.S. Cl. CPC G06T 7/0012; G16H 50/20; G06T 2207/10088; G06T 2207/20081; G06T 2207/30016

(57) ABSTRACT

A system and method of predicting a condition of a fetus may include receiving a magnetic resonance imaging (MRI) scan of the fetus, comprising a sequence of slices; detecting a volume of interest (VOI) representing a location of a brain of the fetus; segmenting one or more slices comprised in the VOI to a set of brain structures selected from a right hemisphere, a left hemisphere, a right lateral ventricle, and a left lateral ventricle; based on said segmentation, calculating at least one ventricle; and predicting a condition of the fetus based on the at least one ventricle metric.

### CROSS-REFERENCE TO RELATED APPLICATIONS

This application is a ByPass Continuation of PCT Patent Application No. PCT/IL2022/050204 having International filing date of Feb. 21, 2022, which claims the benefit of U.S. Patent Application No. 63/151,739, filed Feb. 21, 2021, and entitled “METHODS FOR FETAL EVALUATIONS, PREGNANCY MANAGEMENT AND PREDICTING MODE OF DELIVERY”, which is hereby incorporated by reference in its entirety.

### FIELD OF THE INVENTION

The present invention relates generally to the field of assistive diagnostics. More specifically, the present invention relates to a method and system for evaluating or predicting a condition of a fetus.

### BACKGROUND OF THE INVENTION

Fetal development is a complex process that includes significant changes in unique spatial-temporal trajectories. About 15% of pregnancies are at risk for various developmental disorders, including low birthweight, pre-term birth, congenital heart disease, malformation and congenital anomalies, infections and genetic mutations, intrauterine Growth Restriction (IUGR), infections and injury (e.g., cytomegalovirus infection), various genetic mutations and chromosomal abnormalities, and/or macrosomia (enlarged fetus).

Ultrasound (US) is currently the primary imaging modality for fetal assessment. New developments in ultrasound technology, including color and power Doppler, transvaginal sonography and 3/4D imaging are used to enhance assessment of fetal growth, early diagnose of anomalies and developmental disorders, and assessment of placental insufficiency.

There are many cases of misdiagnosis with severe short and long-term consequences for the fetal and maternal health. Up to 20% of cases diagnosed using ultrasound were shown to have false or misdiagnosis. The highest misclassification rates occur for CNS anomalies and cardiac malformation. Such cases are also related to pathological findings. A recent review study reported that approximately 22% of fetal anomalies are missed by ultrasound, but can be detected with autopsy.

In-vivo fetal imaging and accurate quantitative assessment of fetal organs are of paramount importance in evaluating and determining the development of the fetus. Although ultrasound is the primary imaging modality for fetal assessment, yet it is limited by various factors leading to low diagnostic sensitivity and late or misdiagnosis. Magnetic Resonance Imaging (MRI) is increasingly used to confirm/exclude suspected findings on US, and to provide additional structural and functional information.

Fetal MRI has been used for more than 30 years in clinical setup. The safety of MRI use in pregnant women has been confirmed, making fetal MRI increasingly widespread in cases of unclear ultrasound findings, to confirm/reject suspected abnormalities, and to detect additional abnormalities. MRI has significantly superior capabilities in comparison with ultrasound including 3D information, multiple image contrasts relating to different micro-properties of the tissue, morphologic, functional, and metabolic characterization.

Recent studies show that fetal MRI optimizes perinatal management in up to 30% of cases initially evaluated with US. However, the widespread use of fetal MRI diagnosis is hampered by the need for a specialized MRI scanning protocol, by the shortage of expert fetal MRI radiologists, by the need to manually extract morphological measures and by the limited imaging biomarkers available for various prenatal disorders. Clinical practice in fetal imaging is lags behind other fields in MRI (such as brain and cardiac), not taking full advantage of its capabilities. Fetal MRI is based largely on structural information, often with no use of advanced MRI methods. In addition, despite growing evidence for the importance of normal placental function for fetal as well as maternal health, there is no evolution of the placental function using MRI and using US functional assessment is indirect and restricted to blood vessels outside the placenta. Interpretation of fetal MRI is subjective and mostly qualitative with only a few manually extracted bi-dimensional quantitative measures.

The placenta is an essential organ for normal development of the fetus. Placental insufficiency, e.g., impairment of the placental function, is most frequently represented by fetal growth restriction (FGR), and carries an increased risk for neonatal, childhood and adulthood morbidity and mortality. Several placental functional and structural parameters are known to characterize normal placental development and placental insufficiency. Uterine Artery Doppler Ultrasound, which is the method of choice in obstetrics, providing indirect information regarding blood flow within the umbilical cord, detects abnormal flow in cases with placental insufficiency.

Placental volumes were shown to increase with gestational age (GA), while smaller placental volumes and marginal umbilical cord insertion were found to be associated with abnormal placental function. Using advanced MRI methods, several studies characterized changes in placental perfusion using Arterial-Spin-Labeling (ASL) with GA, reporting inconsistent results. Despite these methodological advancements and knowledge, it is still difficult to diagnose placental insufficiency before it affects the fetus.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods which are meant to be exemplary and illustrative, not limiting in scope.

In one aspect provided herein is a method comprising: receiving a plurality of fetal magnetic resonance imaging (MRI) scans, each comprising a sequence of scan slices; segmenting each of the scan slices regions associated with one or more fetal tissue types, wherein the segmenting associated each voxel in each of the scan slices with one of the one or more fetal tissue types; at a training stage, training a machine learning (ML) model on a training set comprising: (i) all of the scan slices comprising the segmenting, and (ii) labels associated with each tissue type represented in the regions; and at an inference stage, applying the trained ML model to a target scan slice from a target fetal MRI scan of a target fetus, to determine an association between each voxel in the target scan slice and one of the fetal tissue types.

In a further aspect, applying comprises applying the trained ML model to all target slice scans in the target fetal MRI scan, to determine an association between each voxel in the target fetal MRI scan and one of the fetal tissue types.

In a further aspect, the invention includes determining a total weight of the target fetus, based on the associating and a relative density of each of the tissue types.

In a further aspect, fetal tissue types may relate to tissues and/or organs such as a brain, ocular, cardiac, fat, muscles, or any combination thereof.

In a further aspect, tissue types comprise brain right hemisphere, brain left hemisphere, cerebellum, brain stem, brain right lateral ventricle, brain left lateral ventricle, CSF, or any combination thereof.

In a further aspect, tissue types comprise orbit, lens, globe segmentation or any combination thereof.

In a further aspect provided herein is a method comprising: receiving a magnetic resonance (MR) scan of a placenta; segmenting, the placenta in the MR scan, to determine (a) a volume of the placenta; segmenting, in the MR scan, a vascular tree of the placenta, to determine one or more of: (b) cord insertion location, (c) number of bifurcations, and (d) blood vessel dimensions; applying a statistical covariance analysis to two or more of (a), (b), (c), and (d), to predict a birthweight of a fetus associated with the placenta, based, at least in part, on known correlations between (a), (b), (c), and (d) and fetal birthweight.

In a further aspect, segmenting further determines vascular parameters such as blood volume, flow, resistance, or both.

In a further aspect, scan is in-vivo scan.

In a further aspect, segmenting further determines placental insufficiency, placental structure, placental function, or any combination thereof.

In a further aspect provided herein is a method comprising: receiving, as input, a plurality of fetal magnetic resonance imaging (MRI) scans, each comprising a sequence of scan slices; assigning a score to each of the plurality of fetal MRI scans, based on parameters associated with: symmetry in each of the fetal MRI scans, image quality of each of the fetal MRI scans, and object movement; selecting, based on the assigning, a reference fetal MRI scan from the plurality of fetal MRI scans; applying a trained ML model to select a reference slice from the selected reference fetal MRI scan, wherein the ML model is trained to classify scan slices based on a usability parameter with respect to a specified measurement to be performed in the selected scan slice.

In a further aspect, at a training stage, the trained ML model is trained on a training set comprising: (i) a plurality of scan slices associated with a plurality of fetal MRI scans; and (ii) labels associated with a usability parameter with respect to a specified measurement to be performed in scan slices.

In a further aspect, the specified measurement is one or more of: Cerebral Biparietal Diameter (CBD), Bone Biparietal Diameter (BBD), Trans-Cerebellum Diameter (TCD), front occipital diameter (FOD), Vermian Height (VH), and Lateral Ventricle Width.

In a further aspect, the specified measurement is one or more of: binocular (BOD), interocular (IOD), ocular (OD), lens aligned (OD-LA-OD), or any combination thereof.

In a further aspect, the invention includes assessing the reliability of the CBD the BBD measurements, comprising computing the measurement values on the reference slice, wherein if computed values on two images are higher than a reliability threshold, a CBD/BBD measurement reliability warning is issued.

In a further aspect, computing the measurement values on the reference slice comprises the use of an image processing technique for improving contrast in the images.

In addition to the exemplary aspects and embodiments described above, further aspects and embodiments will become apparent by reference to the figures and by study of the following detailed description.

Embodiments of the invention may include a method of predicting a condition of a fetus by at least one processor. Embodiments of the method may include: receiving a magnetic resonance imaging (MRI) scan of the fetus, that may include a sequence of slices; detecting a volume of interest (VOI) representing a location of a brain of the fetus; segmenting one or more slices comprised in the VOI to a set of brain structures selected from a right hemisphere, a left hemisphere, a right lateral ventricle, and a left lateral ventricle; based on said segmentation, calculating at least one ventricle metric, selected from: (i) a right lateral ventricle volume, (ii) a left lateral ventricle volume, (iii) an average of volumes of the right and left ventricles, and (iv) asymmetry between volumes of the right and left lateral ventricles; and predicting a condition of the fetus based on the at least one ventricle metric.

According to some embodiments, the condition of the fetus may include, for example ventriculomegaly, macrocephaly and microcephaly.

Embodiments of the invention may include a method of automatically annotating at least one brain structure in an MRI scan by at least one processor. Embodiments of the method may include: receiving an MRI scan of a fetus, may include a sequence of slices; detecting a VOI representing a location of a brain of the fetus, depicted in the scan; identifying a first anatomic location in a first slice of the sequence of slices, within the VOI; identifying at least two second anatomic locations in at least one second slice of the sequence of slices within the VOI; and annotating at least one brain structure depicted in the VOI, based on a relative positioning of said identified anatomic locations.

Additionally, or alternatively, the at least one processor may be configured to: apply a first ML model on the first slice and on the at least one second slice, wherein said first ML model may be trained to segment each slice of the first slice and the at least one second slice to a plurality of segments. The at least one processor may subsequently identify the anatomic locations by identifying specific segments of the plurality of segments as representing specific brain structures of a predefined set of brain structures.

Additionally, or alternatively, the at least one processor may determine a direction of the sequence of slices based on said identification of segments; and annotate the at least one brain structure by applying a label to the one or more segments. The label may represent pertinence to a left-side brain structure or a right-side brain structure, based on the determined direction.

Additionally, or alternatively, the label may be selected from a set of labels consisting of: a right hemisphere, a left hemisphere, a right lateral ventricle, a left lateral ventricle, a cerebellum, a right eye, and a left eye.

Additionally, or alternatively, the at least one processor may be configured to identify the first anatomic location and the at least two second anatomic locations by applying a second ML model on the VOL The second ML model may be trained to identify three or more anatomic locations that may be landmarks in the brain, as depicted in slices comprised in the VOI.

Additionally, or alternatively, the at least one processor may be configured to: determine a direction of the sequence of slices based on said identification of three or more landmarks; and annotate the at least one brain structure by applying a label to at least one scan of the sequence of scans. The label may represent a left-right orientation of the depicted brain structure.

Embodiments of the invention may include a method of predicting a condition of a fetus by at least one processor. Embodiments of the method may include: receiving an Mill scan of the fetus, may include a sequence of slices; detecting a VOI representing a location of a brain of the fetus depicted in the scan; applying at least one ML model on the VOI, to identify two or more landmarks depicted in the scan; calculating at least one distance between the two or more landmarks; and predicting the condition of the fetus based on the calculated at least one distance.

According to some embodiments, the at least one distance may be a cranial distance such as a Cerebral Biparietal Diameter (CBD), a Bone Biparietal Diameter (BBD), a Trans-Cerebellum Diameter (TCD), a Front Occipital Diameter (FOD), a Vermian Height (VH), and a Lateral Ventricle Width.

According to some embodiments, the at least one distance may be an ocular distance such as a Binocular Distance (BOD), an Interocular Distance (IOD), an Ocular Distance (OD), and a Lens Aligned Distance (OD-LA-OD).

According to some embodiments, the fetal condition may include, for example hypertelorism or hypotelorism.

Additionally, or alternatively, the at least one processor may be configured to: apply a first ML model on one or more slices of the sequence of slices, to produce one or more respective slice scores; and select a reference slice from the one or more slices, based on the respective slice scores. The first ML model may be trained to produce the slice score for each specific slice as a prediction of a probability of selection of the relevant slice by an expert, for the purpose of measuring a specific distance.

Additionally, or alternatively, the at least one processor may be configured to apply a second ML model on a subset of the sequence of slices that may include the reference slice, to perform multi-class segmentation of the slices to fetal brain structures. The second ML model may be trained to: segment each slice to a plurality of segments; and identify each of the segments as representing a brain structure of a predefined set of brain structures. The predefined set of brain structures may include, for example a right hemisphere, a left hemisphere, and a cerebellum.

Additionally, or alternatively, the at least one processor may be configured to calculate a midsagittal line in the reference slice, based on said multi-class segmentation; calculate a brain orientation vector in the reference slice, based on said multi-class segmentation; and identify the two or more landmarks based on the midsagittal line and the brain orientation vector.

Embodiments of the invention may include a method of predicting a condition of a fetus by at least one processor. Embodiments of the method may include: receiving an MRI scan of a womb that may include a sequence of slices; applying at least one first ML model on one or more slices of the sequence of slices to segment a placental VOI, representing a placenta depicted in the Mill scan; calculating a volume of said placental VOI; identifying an umbilical cord insertion location in said placental VOI; calculating an umbilical cord score, representing a marginality of the umbilical cord insertion location in said placenta; and predicting the condition of the fetus based on the calculated placental volume and umbilical cord score.

According to some embodiments, the condition of the fetus may include, for example Fetal Growth Restriction (FGR), placental insufficiency, and placental dysfunction.

Additionally, or alternatively, the at least one processor may be configured to: apply at least one second ML model on the placental VOI, to obtain at least one vascular metric value; and predict the condition of the fetus further based on the at least one vascular metric value.

According to some embodiments, the second ML model may be trained to predict the at least one vascular metric value based on the placental VOL The at least one vascular metric may be selected from a list consisting of: Placental Blood Flow (PBF) and Arterial Transit Time (ATT).

Embodiments of the invention may include a system for predicting a condition of a fetus. Embodiments of the system may include: a non-transitory memory device, wherein modules of instruction code may be stored, and at least one processor associated with the memory device, and configured to execute the modules of instruction code.

Upon execution of said modules of instruction code, the processor may be configured to: receive a magnetic resonance imaging (Mill) scan of the fetus, may include a sequence of slices; detect a volume of interest (VOI) representing a location of a brain of the fetus; segment one or more slices comprised in the VOI to a set of brain structures selected from a right hemisphere, a left hemisphere, a right lateral ventricle, and a left lateral ventricle; based on said segmentation, calculate at least one ventricle metric, selected from: (i) a right lateral ventricle volume, (ii) a left lateral ventricle volume, (iii) an average of volumes of the right and left ventricles, and (iv) asymmetry between volumes of the right and left lateral ventricles; and predict a condition of the fetus based on the at least one ventricle metric.

Additionally, or alternatively, upon execution of said modules of instruction code, the processor may be configured to: receive an MRI scan of the fetus that may include a sequence of slices; detect a VOI representing a location of a brain of the fetus depicted in the scan; apply at least one ML model on the VOI, to identify two or more landmarks depicted in the scan; calculate at least one distance between the two or more landmarks; and predict the condition of the fetus based on the calculated at least one distance.

Additionally, or alternatively, upon execution of said modules of instruction code, the processor may be configured to: receive an MRI scan of a womb, may include a sequence of slices; apply at least one first ML model on one or more slices of the sequence of slices to segment a placental VOI, representing a placenta depicted in the MRI scan; calculate a volume of said placental VOI; identify an umbilical cord insertion location in said placental VOI; calculate an umbilical cord score, representing a marginality of the umbilical cord insertion location in said placenta; and predict the condition of the fetus based on the calculated placental volume and umbilical cord score.

Embodiments of the invention may include a system for automatically annotating at least one brain structure in an MRI scan. Embodiments of the system may include: a non-transitory memory device, wherein modules of instruction code may be stored, and at least one processor associated with the memory device, and configured to execute the modules of instruction code.

Upon execution of said modules of instruction code, the processor may be configured to: receive an MRI scan of a fetus, may include a sequence of slices; detect a VOI representing a location of a brain of the fetus, depicted in the scan; identify a first anatomic location in a first slice of the sequence of slices, within the VOI; identify at least two second anatomic locations in at least one second slice of the sequence of slices within the VOI; and annotate at least one brain structure depicted in the VOI, based on a relative positioning of said identified anatomic locations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for evaluating or predicting a condition of a fetus according to some embodiments;

FIG. 2 is a block diagram, depicting an example of an application of a system for evaluating or predicting a condition of a fetus, as part of an assistive diagnostic pipeline, according to some embodiments;

FIG. 3 is a block diagram, depicting modules of a system for evaluating or predicting a condition of a fetus, according to some embodiments;

FIG. 4 is a schematic diagram depicting a process of segmenting scan slices to a plurality of segments representing brain structures, according to some embodiments of the invention;

FIG. 5 depicts four examples of fetal MRI scans, adjoint with brain structure segmentation and corresponding diagnoses, as provided by embodiments of the invention;

FIG. 6 is a flow diagram depicting a method of estimating fetal weight based on volumetric scans, according to some embodiments of the present invention;

FIG. 7 is a flow diagram depicting a method of estimating fetal weight according to pixel/voxel intensity values as presented in fetal volumetric scans, according to some embodiments of the present invention;

FIG. 8 is a flow diagram depicting a method of training an ML model to classify tissue types in fetal volumetric scans, according to some embodiments of the present invention;

FIG. 9 is a schematic diagram depicting a method of performing cerebral fetal MRI biometric measurements, according to some embodiments of the invention;

FIG. 10 is a schematic diagram depicting a process of scan slice selection, according to some embodiments of the invention;

FIG. 11 is a flow diagram depicting an example of a method of selecting an optimal volumetric scan and/or a volumetric scan slice, according to embodiments of the present invention;

FIG. 12 is a flow diagram depicting an example of a method of training an ML model to optimally select volumetric scan slices, according to some embodiments of the present invention;

FIGS. 13A and 13B are images depicting steps in a method of performing cerebral fetal MRI biometric measurements, according to some embodiments of the invention;

FIGS. 14A, 14B and 14C are images depicting steps in another method of performing cerebral fetal MRI biometric measurements, according to some embodiments of the invention;

FIGS. 15A and 15B are images depicting steps in another method of performing cerebral fetal MRI biometric measurements, according to some embodiments of the invention;

FIGS. 16A and 16B are images depicting two fetal MRI biometric measurements (e.g., TCD images), and evaluation of reliability of the measurements, according to some embodiments of the invention;

FIG. 17 is a flow diagram showing offline training (left) and online inference (right) phases of a method of performing cerebral fetal MRI biometric measurements, according to some embodiments of the invention;

FIG. 18 is a schematic diagram depicting a method of performing ocular fetal MRI biometric measurements, according to some embodiments of the invention;

FIGS. 19A and 19B are images depicting two-dimensional (2D) fetal ocular measurements on a representative fetal MRI scan slice, as provided by embodiments of the invention;

FIG. 20A and FIG. 20B are images depicting T1-weighted MR images of two normal placentas;

FIGS. 21A, 21B and 21C are images depicting segmentation of a placenta, a fetus body and fetal brain respectively, as calculated by embodiments of the invention;

FIG. 22A is an anatomical image depicting a representative T2 scan slice of a womb, accommodating a fetus and a placenta, at a Gestational Age (GA) of 32 weeks;

FIG. 22B is an image depicting values of Placental Blood Flow (PBF), superimposed over the anatomical image of FIG. 22A, as calculated by embodiments of the invention;

FIG. 22C is an image depicting values of Arterial Transit Time (ATT), superimposed over the anatomical image of FIG. 22A, as calculated by embodiments of the invention; and

FIGS. 23A, 23B and 23C are flowcharts of methods of predicting a condition of a fetus according to some embodiments of the present invention; and

FIG. 24 is a flowchart of a method of automatically annotating at least one brain structure in an MRI scan according to embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

As used herein, the terms “volumetric imaging” or “volumetric scans” may refer to techniques and processes for creating visual representations of internal anatomical structures. Examples of volumetric imaging techniques include, among others, magnetic resonance imaging (MRI), positron emission tomography (PET), and the like. Volumetric imaging techniques typically produce an image dataset, also referred to herein as a “scan” or “volumetric scan”. These volumetric scans may include a series or a sequence of two-dimensional (2D) cross-sectional images, acquired through the scanned volume, and referred to herein as “slices” or “scan slices”.

As known in the art, the slices may be individually, or cumulatively analyzed, for example to construct a three-dimensional (3D) volume representing a structure of the scanned object.

As used herein, the terms “image segmentation” or “segmentation” may refer to the process of partitioning an image into different meaningful segments, which may correspond to, or represent different tissue classes, organs, pathologies, and/or other biologically-relevant structures, for example through a binary classification of pixels or voxels in an image.

As elaborated herein, based on a set of MRI slices, the anatomy of a body part may be segmented. The segmentation process may include classifying the pixels or voxels of a slice into a predetermined number of classes that are homogeneous with respect to some characteristic (e.g., intensity, texture, MRI parameter values, etc.).

For example, in a segmented image of a fetus, the anatomy of the fetus can be categorized into two or more classes, based for example on tissue type, such as muscle tissue, adipose tissue, bone tissue, fluid (e.g., blood), etc. Once the segmented image is generated, it can be used for different purposes. For example, the segmented image slices may be merged to form a volume segmented into sub-volumes based on tissue type.

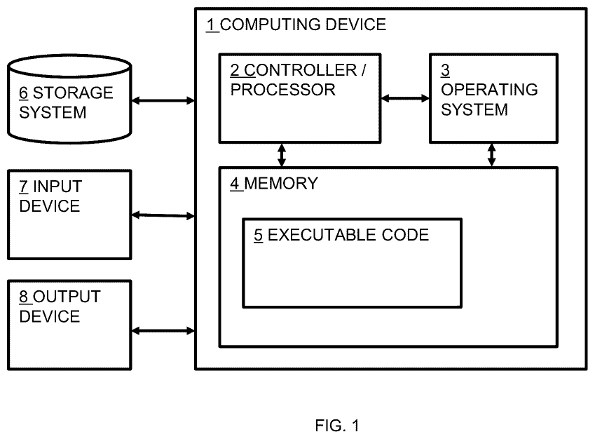

Reference is now made to FIG. 1, which is a block diagram depicting a computing device, which may be included within an embodiment of a system for evaluating or predicting a condition of a fetus, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may evaluate or predict a condition of a fetus, as further described herein.

Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to a condition of a fetus may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse, and the like. Output devices 8 may include one or more (possibly detachable) displays or monitors, speakers and/or any other suitable output devices. Any applicable input/output (I/O) devices may be connected to computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

A neural network (NN) or an artificial neural network (ANN), e.g., a neural network implementing a machine learning (ML) or artificial intelligence (AI) function, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

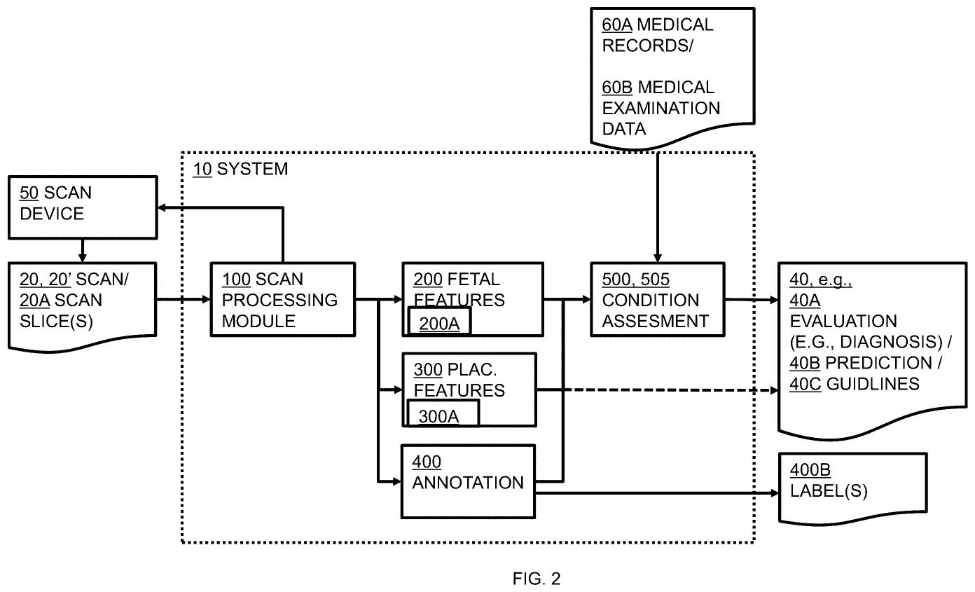

Reference is now made to FIG. 2, which is a block diagram depicting an example of an application of a system 10 for evaluating or predicting a condition of a fetus, as part of an assistive diagnostic pipeline, according to some embodiments.

According to some embodiments of the invention, system 10 may be implemented as a software module, a hardware module, or any combination thereof. For example, system 10 may be or may include a computing device such as element 1 of FIG. 1, and may be adapted to execute one or more modules of executable code (e.g., element 5 of FIG. 1) to evaluate or predict a condition of a fetus, as further described herein.

As shown in FIG. 2, system 10 may be integrated into a pipeline of assistive diagnosis. As elaborated herein, system 10 may be configured to receive images, such as fetal and/or maternal MRI scans 20 or scan slices 20A from a scanning device 50 (e.g., an MRI scanner).

According to some embodiments, system 10 may be implemented on a platform that is separate from scanning device 50 (e.g., MRI scanner), and may be communicatively connected to scanning device 50 via a communication network (e.g., the Internet). Additionally, or alternatively, system 10 may be implemented on the same platform (e.g., the same computing device) as scanning device 50, and may utilize the computing resources of scanning device 50.

System 10 may include a scan processing module 100, adapted to apply a variety of image processing algorithms on the received scans 20, and collaborate with fetal features module 200 and/or placental features module 300 to extract one or more features or parameters (200A, 300A, respectively) from the scans 20 or scan slices 20A.

As shown in FIG. 2, system 10 may include a condition assessment module 500 (or “condition module 500” for short). Condition module 500 may be adapted to receive features or parameters 200A, 300A, in addition to optional relevant information. The additional optional information may be, for example clinical, genetical and/or and lab test information of the fetus and/or mother, originating from medical records 60A and/or medical examination data 60B. Condition module 500 may be configured to produce a report or notification 40 based on the received information (200A, 300A, 60A, 60B), and may transmit report 40 e.g., as an electronic message, such as an email, to a predefined destination (e.g., an email address of a physician or care giver).

As elaborated herein, report 40 may include for example an evaluation or diagnosis 40A of a current condition of the fetus and/or mother. Evaluation or diagnosis 40A may include, for example a condition relating to the fetus' growth, such as a condition of Fetal Growth Restriction (FGR). In another example, evaluation or diagnosis 40A may include a condition relating to the fetus' brain, such as Ventriculomegaly, macrocephaly, microcephaly, and the like. In another example, evaluation or diagnosis 40A may include an assessment or measurement of a structure or organ of the fetus, mother, or placenta, including for example cerebral measurements, ocular measurements, placental measurements, fetus body volume, fetus body weight, and the like.

Additionally, or alternatively, and as elaborated herein, report 40 may include a prediction 40B of a future condition of the fetus, also referred to herein as predicted newborn outcomes. Such predictions 40B may include, for example an expected weight at childbirth, expected evolution of a suspected condition related to the fetus body (e.g., FGR), expected evolution of a suspected condition related to the brain (e.g., ventriculomegaly, macrocephaly, microcephaly), expected evolution of a suspected condition relating to the placenta (e.g., placental dysfunction), and the like.

Additionally, or alternatively, report 40 may include guidelines 40C for pregnancy management, based on diagnosis 40A and/or prediction 40B. For example, guidelines 40C may include a guideline for optimal gestation period (e.g., an optimal time for delivery) based on placental insufficiency parameters and placental insufficiency's effect on fetal growth and organs' function, a recommended in-utero intervention, a recommended newborn developmental therapy (such as brain reorganization following brain insult), and the like, in order to improve neonatal outcome.

As shown in FIG. 2, system 10 may include condition module 500. For example, condition module 500 may be implemented by the same computing device(s) as modules 100, 200, 300 and/or 400. Additionally, or alternatively, condition assessment module 500 may be a third-party module of assistive diagnosis, and may be communicatively connected to system 10 by a communication network (e.g., the Internet, a cellular data network, and the like).

As known in the art, single scan slices are devoid of clear annotation or indication regarding the direction or orientation of a scan. For example, when examining an image of a transverse plane scan slice of a brain, physicians often resort to guessing which side of the image depicts the right brain hemisphere, and which depicts the left.

According to some embodiments of the invention, and as elaborated herein, system 10 may include an annotation module, adapted to automatically provide annotations or labels 400B regarding structures depicted in slices 20A. Pertaining to the example above, annotations or labels 400B may include, for example labels of segmented structures, adjoint with their respective direction or laterality, such as “right hemisphere”, “left hemisphere”, “right eye”, “left eye”, “right lateral ventricle”, “left lateral ventricle”, and the like.

arrows may represent flow of one or more data elements to and from system 10 and/or among modules or elements of system 10. Some arrows have been omitted in FIG. 2 for the purpose of clarity.

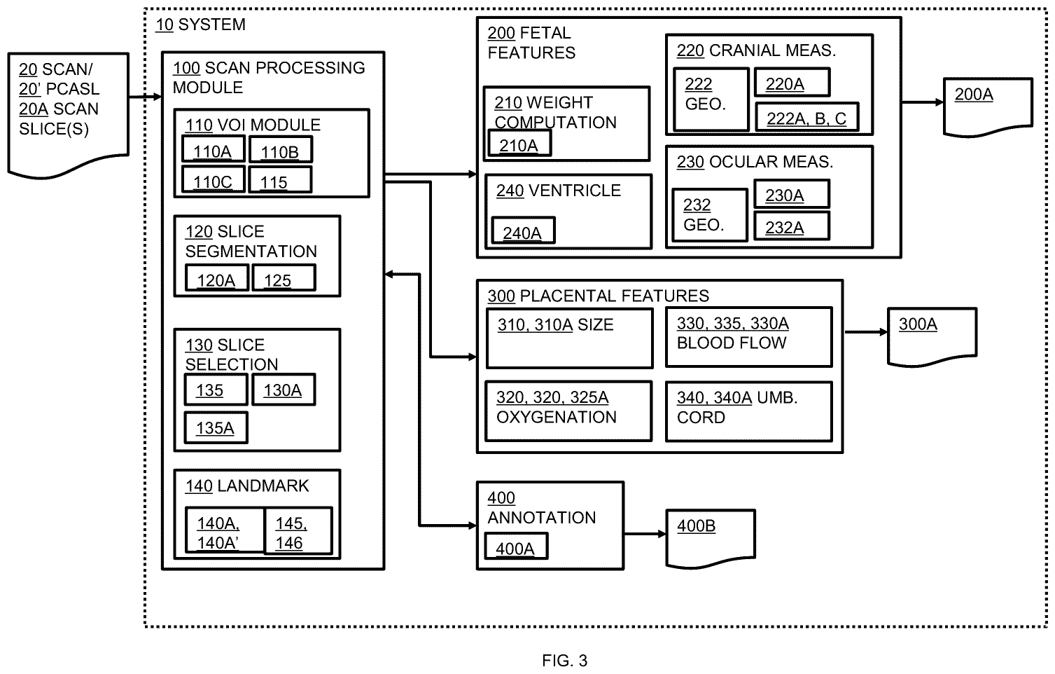

Reference is now made to FIG. 3, which is a block diagram depicting modules of system 10 for evaluating or predicting a condition of a fetus, according to some embodiments. System 10 of FIG. 3 may be the same as system 10 of FIG. 2.

As shown in FIG. 3, scan processing module 100 (or “module 100”, for short) may receive a magnetic resonance imaging (MRI) scan 20 of a fetus, including a plurality or sequence of slices 20A.

According to some embodiments, module 100 may include a volume of interest (VOI) detection module 110, configured to detect or identify a brain VOI 110A in scan 20, that includes, or represents a location of a brain of the fetus.

In some embodiments, VOI detection module 110 may be, or may include a machine-learning (ML) based model, such as a convolutional neural network (CNN), trained to detect, or spatially segment brain VOI 110A from scan 20.

For example, VOI detection module 110 may be, or may include a 3D or 2D two-stage anisotropic U-Net model, that may be trained to segment a volume of a brain from scan 20.

As commonly referred to in the art, the term “U-Net” may refer to a type of CNN, that may be used for a variety of applications. For example, 2D U-Net models may be used to perform analysis functions on 2D input data, such as image segmentation. Similarly, 3D U-Net models may be used to analyze 3D input data structures, such as volumetric scan 20.

As known in the art, a U-Net model typically includes a first portion, which may be referred to herein as an encoder, and a second portion, which may be referred to herein as a decoder. The encoder may be adapted to receive an input data structure such as a 3D scan (for 3D U-Nets) or a 2D slice (for 2D U-Nets) to be analyzed. The encoder may be trained to produce a representation of the input data structure, in a latent feature space that is reduced in dimension in relation to the received data. The decoder may be trained to produce a reconstructed version of the input data structure.

Pertaining to the current example, VOI detection module 110 may receive a volumetric scan 20 as input data, the encoder side of VOI detection module 110 may encode volumetric scan 20 in a latent space of reduced dimension (e.g., having a number of components that is smaller than the number of voxels of volumetric scan 20), and the decoder side of VOI detection module 110 may produce a reproduced version of volumetric scan 20, that includes a segmentation, or identification of brain VOI 110A. It may be appreciated that additional ML architectures, such as deep CNN types and models may also be used.

According to some embodiments, VOI detection module 110 may then proceed to crop brain VOI 110A as an axis-aligned, 3D bounding box of the resulting segmentation.

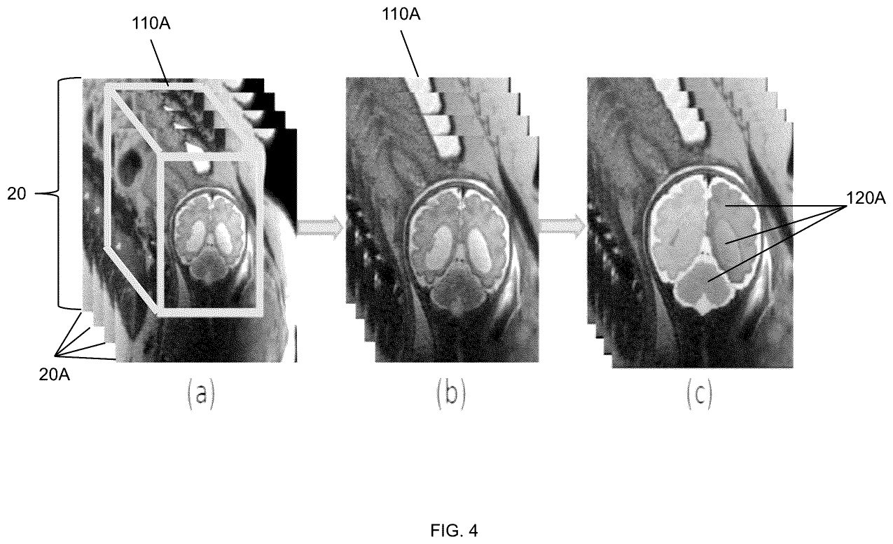

Reference is also made to FIG. 4, which is a schematic diagram depicting a process of segmenting scan slices to a plurality of segments representing brain structures, according to some embodiments of the invention. As shown in FIG. 4 (a), VOI detection module 110 may detect brain VOI 110A in original scan 20. As shown in FIG. 4 (a), VOI detection module 110 may then crop brain VOI 110A as an axis-aligned, 3D bounding box from scan 20. The term “axis aligned” may be used in this context to indicate alignment of brain VOI 110A to the axes of original scan 20.

As shown in FIG. 3, module 100 may include a slice segmentation module 120, configured to segment one or more slices 20A comprised in brain VOI 110A to a set of brain structures.

In other words, slice segmentation module 120 may be trained to produce, from one or more slices 20A, one or more segments 120A. Each such segment 120A may include a plurality of pixels or voxels of the relevant slice 20A that pertain to a specific depicted organ or structure.

According to some embodiments, slice segmentation module 120 may be, or may include an ML-based model, such as a CNN model, adapted to segment regions of an image, as known in the art.

For example, slice segmentation module 120 may be, or may include an ML model 125 such as a 2D U-Net CNN model, that includes an encoder portion (e.g., a 34-layer Residual Network (Resnet34) encoder) and a decoder portion. During a pre-training stage, the U-Net model may be pre-trained on a training dataset (e.g., the ImageNet database), using a loss function such as the Lovasz loss function. The training period may be split between the encoder and decoder. For example, during a training period of 24 epochs, in the first 12 epochs only the decoder layers may be trained, and in the subsequent 12 epochs, both the encoder and decoder layers may be trained.

In a subsequent testing stage (also referred to herein as an “offline inference stage”), various processes of data augmentation may be applied to the training dataset. Such data augmentation may include random rotation, application of intensity inhomogeneity filters, random horizontal and/or vertical flips, brightness adjustments, contrast adjustments, and the like.

In a subsequent inference stage, slice segmentation module 120 (e.g., slice segmentation ML model 125) may be applied on target slices 20A, such as the ones depicted in FIG. 4.

For example, as depicted in FIG. 4 (c), slice segmentation module 120 (e.g., slice segmentation ML model 125) may be configured to produce segments 120A that represent structures such as a right hemisphere (red), a left hemisphere (azure), a cerebellum and brain stem (green), a right lateral ventricle (blue), a left lateral ventricle (pink) and cerebrospinal fluid (CSF, yellow). It may be noted that identification of any of segments 120A as specifically representing either a left-hand side structure or a right-hand side structure (e.g., right or left ventricle, right or left hemisphere, etc.) may be performed in collaboration with annotation module 140, as elaborated herein.

Additionally, during a subsequent post-processing stage, segmentation module 120 may be configured to apply image processing algorithms to one or more segments 120A of one or more slices 20A. For example, segmentation module 120 may remove small objects (e.g., areas represented by a number of pixels that is below a predefined threshold) that are connected to segments 120A. In another example, segmentation module 120 may remove large, connected components (e.g., areas represented by a number of pixels that surpasses a predefined threshold), which do not overlap with corresponding areas of adjacent slices 20 in the sequence of slices.

According to some embodiments, fetal features module 200 may be configured to receive the one or more segmented 120A slices 20A, and may produce therefrom one or more properties or features (e.g., 200A of FIG. 2), representing a condition of the fetus.

For example, fetal features module 200 may include a ventricles feature extraction module 240 (or “module 240” for short), configured to calculate at least one ventricle feature value or ventricle metric value 240A, based on the segmentation of slices 20A to segments 120A. For example, fetal features module 200 may accumulate or summarize areas of segments 120A of a plurality of slices 20A, to produce ventricle metric values 240A such as (i) a right lateral ventricle volume, (ii) a left lateral ventricle volume, (iii) an average of volumes of the right and left ventricles, and (iv) a metric representing asymmetry between volumes and/or shapes of the right and left lateral ventricles.

According to some embodiments, condition module 500 of FIG. 2 may be configured to produce a prediction 40B or evaluation 40A of a condition of the fetus based on the at least one ventricle metric 240A value.

For example, condition module 500 may receive, as part of the medical records 60A information, such as a GA of the fetus, a gender of the fetus, maternal physical information such as Body Mass Index (BMI), data pertaining to maternal medical history (e.g., previously diagnosed maternal conditions or diseases) and genetic information. Condition module 500 may apply rule-based prediction or evaluation of a condition of the fetus based on the ventricle metric 240A value and the medical records 60A (e.g., GA) information.

For example, condition module 500 may compare a ventricle metric 240A value that is a volume (e.g., in voxels, in cubic millimeters, etc.) of a lateral ventricle to an expected, normal range of volumes at the relevant GA 60A. Condition module 500 may subsequently produce an evaluation 40A as a warning, in case that the current measurement (e.g., the lateral ventricle volume) exceeds the normal range of volumes, a fetal condition commonly referred to as Ventriculomegaly.

In another example, condition module 500 may produce an evaluation 40A as a warning, in case that the ventricle metric 240A value (e.g., the lateral ventricle volume) falls short of the normal range of volumes.

In another example, condition module 500 may produce an evaluation 40A as a warning, in case that the ventricle metric 240A value (e.g., the lateral ventricle volume asymmetry) surpasses a predefined threshold.

In another example, fetal features module 200 may calculate a metric of hemisphere asymmetry, e.g., quantifying a level of asymmetry between the right hemisphere segment 120A and left hemisphere segment 120A. Condition module 500 may subsequently produce an evaluation 40A as a warning, in case that the metric of hemisphere asymmetry surpasses a predefined threshold.

In another example, condition module 500 may produce an evaluation 40A as a warning, in case that the ventricle metric 240A value (e.g., the lateral ventricle volume average) falls short of a predefined threshold.

In another example, condition module 500 may produce a prediction 40B that may include a predicted value of the fetal features 200A (e.g., the ventricle metric values 240A) based on a combination of the currently measured ventricle metric 240A value and on additional data (60A, 60B) such as the GA 60A and/or previous measurements of fetal features 200A.

In another example, as elaborated herein, fetal features module may include a cranial measurements 220 module, adapted to measure, or calculate at least one distance 220A that is a cranial distance 220A (e.g., CBD, BBD, TCD, and the like). Condition module 500 may receive the one or more cranial distances 220A, and produce an evaluation 40A as a warning, in case that cranial distance 220A is beyond a predetermined range. For example, such an evaluation 40A may include indication of a suspected fetal condition such as macrocephaly, macrocephaly, and the like.

In another example, as elaborated herein, fetal features module may include an ocular measurements 230 module, adapted to measure, or calculate at least one distance 230A that is an ocular distance 230A (e.g., Binocular Distance (BOD), Interocular Distance (IOD), Ocular Distance (OD), and Lens Aligned Distance (OD-LA-OD), and the like). Condition module 500 may receive the one or more ocular distances 230A, and produce an evaluation 40A as a warning, in case that ocular distance 230A is beyond a predetermined range. For example, in cases where IOD is calculated to be beyond a predefined range, system 10 may produce an evaluation 40A that includes indication of a suspected fetal condition such as hypertelorism or hypotelorism.

In yet another example, as elaborated herein, placenta features module 300 may be configured to produce one or more placental features 300A, including for example placental volume 310A, an oxygenation value 320A, a blood flow value 330A, an umbilical cord insertion point value 340A, and the like. Condition assessment module 500 may include one or more ML-based models 505, trained to receive the one or more placental features 300A, and predict one or more respective conditions of the fetus. Such conditions may include, for example Fetal Growth Restriction (FGR), placental insufficiency, placental dysfunction, and the like.

System 10 may train the one or more ML-based models 505 by utilizing supervised training. For example, during a training phase, the one or more ML-based models 505 may be trained based on a training dataset, that may include supervisory data. For example, the training dataset may include a plurality of annotated or labelled scans 20 (or portions of scans 20), depicting the fetus and/or the placenta. The supervisory data (e.g., the annotations) may be introduced by an expert (e.g., a physician or radiologist), and may include information pertaining to a condition of the fetus, such as a physician's diagnosis of FGR, placental insufficiency, placental dysfunction, and the like. Additionally, or alternatively, annotations of the training dataset may include labels of one or more VOIs (e.g., a placental VOI 110A, a fetal body VOI 110A, a fetal brain VOI 110A) as pertaining to a fetal condition (e.g., as an FGR, or non-FGR condition).

It may be appreciated that a wide variety of additional configurations of evaluation 40A and prediction 40B may also possible.

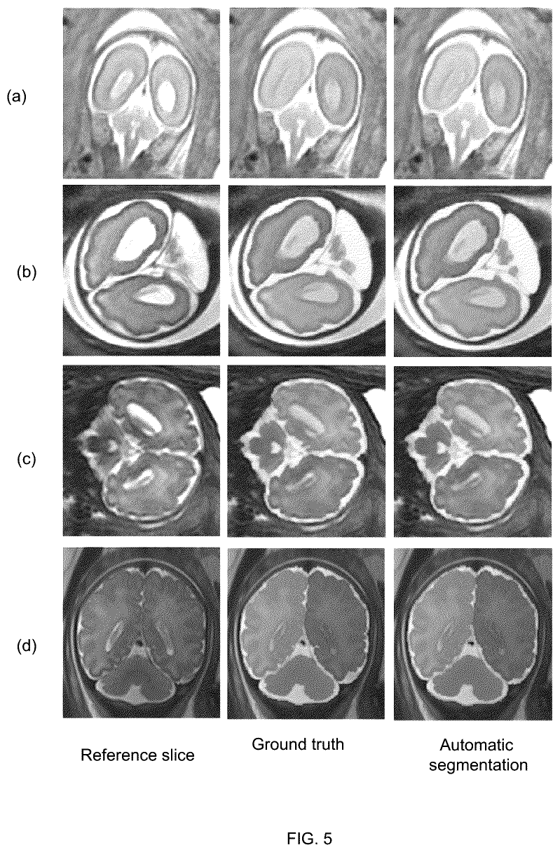

Reference is now made to FIG. 5, which depicts four experimental examples (a-d) of fetal MRI scans (left column), adjoint with ground-truth (e.g., manual) brain structure segmentation (middle column) and brain structure segmentation as provided by embodiments of the invention (right column), with corresponding diagnoses.

By comparing the images of the middle column and right column it may be appreciated that the results of segmentation by slice segmentation module 120 resemble those of the ground-truth.

Additionally, it may be appreciated that diagnoses 40A produced by condition module 500 based on the automated segmentation of slice segmentation module 120 are identical to those provided by a human expert. Example (a) represents a fetal brain at a GA of 22 weeks, and was diagnosed as having normal development. Example (b) represents a fetal brain at a GA 27 weeks, and was diagnosed with multiple cortical malformations. Example (c) represents a fetal brain at a GA of 32 weeks, and was diagnosed with ventriculomegaly (VM). Example (d) represents a fetal brain at a GA of 36 weeks, and was diagnosed as having normal development.

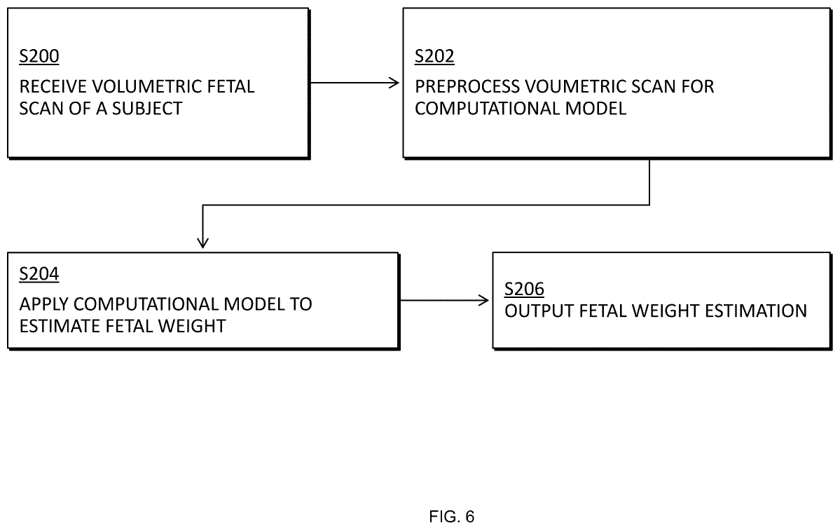

Reference is now made to FIG. 6, which is a flow diagram depicting a method of estimating fetal weight based on volumetric scans, according to some embodiments of the present invention.

According to some embodiments, and as shown in step S200, system 10 may receive a volumetric scan 20 of a subject. For example, volumetric scan 20 may be an MRI scan of a fetus in-situ or in-vivo. Scan 20 may include a series of consecutive two-dimensional (2D) images or slices 20A of the subject (e.g., the fetus).

According to some embodiments, and as shown in step S202, the received volumetric scan 20A may be preprocessed by one or more methods in order to apply one or more computational models to estimate fetal weight and/or fetal birth weight from the preprocessed volumetric scan.

For example, pixel or voxel values of the scan may be viewed or presented on an output device (e.g., element 8 of FIG. 1) such as a screen by different intensity, or grey level values of respective pixels or voxels. These intensity or grey scale values may represent on or more (e.g., a combination of) MRI features and parameters.

Such MRI features and parameters may include for example T1 relaxation time, T2 relaxation time, Proton Density (PD), Apparent Diffusion Coefficient (ADC) values, Fractional Anisotropy (FA), fat and water images, Magnetization Transfer (MT), permeability, and the like.

It may be appreciated that the measured features and parameters represented by may be characteristic of specific tissue types.

According to some embodiments, during a preprocessing or calibration stage, scan processing module 100 may preprocess or calibrate pixel or voxel values of scan 20 such that the grey-level or intensities may represent tissue types of interest.

According to some embodiments, scan processing module 100 may subsequently compare grey-level or intensities with known values that may represent, or be associated with specified tissue types. Based on this comparison, scan processing module 100 may label one or more (e.g., each) pixel or voxel of scan 20 with a numerical probability label 110B.

Numerical probability label 110B (or “label 110B” for short) may represent a certain probability (e.g., in the range between 0 and 1) that the relevant pixel or voxel contains or represents a specific type of tissue. Additionally, or alternatively, the comparison can be made for a group or cluster of pixels or voxels.

According to some embodiments, scan processing module 100 may then segment volumetric scan 20 into one or more sub-volumes 110C based on the labeling 110B of each pixel/voxel, where each sub-volume 110C may be associated with a particular type of tissue.

According to some embodiments, fetal features module 200 of FIG. 3 may include a weight computation module 210, adapted to receive sub-volumes 110C, and their association with particular tissue types.

As shown in step S204, weight computation module 210 may apply a computational model to estimate a weight of the total volume of the fetus, based on the sub-volumes 110C, and their association with particular tissue types. For example, weight computation module 210 may (a) calculate a volume (e.g., in cubic millimeters) of each sub-volume 110C, (b) multiply each calculated volume by a known specific gravity value, associated with each respective tissue type, to obtain the weight of each sub-volume 110C, and (c) accumulate or sum the weight of each sub-volume 110C, to obtain an estimated weight 210A of the fetus depicted in scan 20.

In some embodiments, at step S206, the fetal weight estimation may be output, for example as part of report 40. Report 40 may be sent to a user which may receive the output to a computer screen and/or storage device.

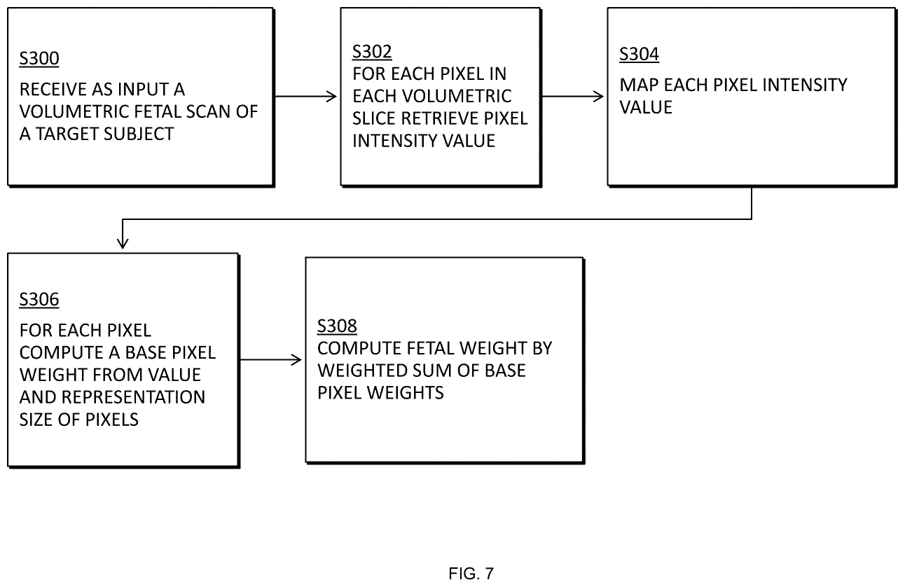

Reference is now made to FIG. 7, which is a flow diagram depicting a method of estimating fetal weight according to pixel/voxel intensity values as presented in fetal volumetric scans, according to some embodiments of the present invention.

As shown in step S300, system 10 may receive a volumetric fetal scan 20 of a target subject, including a sequence of 2D scan slices 20A.

As shown in step S302, scan processing module 100 may calculate an intensity value of each pixel/voxel from each scan slice 20A. It may be appreciated that pixel/voxel intensity values may depend on various factors, such as the configuration of the volumetric scanner which executed the scan, contrasting agents that were administered during the scan and the like. Additionally, pixel/voxel intensity values may correlate to various tissue types as captured within the volumetric scan 20, depending on said factors.

As shown in step S304, scan processing module 100 may map and/or normalize, pixel/voxel intensity values. The mapping may enable quantification of a weight for each pixel/voxel corresponding to a volume of tissue of the fetus as captured in volumetric scan 20. In some embodiments, at step S304, the volumetric scan 20 may relate to a volumetric acquisition or to a 2D acquisition.

As shown in step S306, in some embodiments, for each pixel/voxel a base pixel/voxel weight is computed. Base pixel/voxel weights may be computed from the mapped density value and real-world representation size of pixels by approximating a volume weight according to a tissue type corresponding to the mapped density values, according to steps of: (i) Computing a base volume weight for each pixel/voxel HU scale value (units may comprise mm³×mg); and (ii) for each pixel, scaling a corresponding base volume weight to the volume representation of the pixel.

According to some embodiments, scan processing module may calculate a volume 110C of each tissue type.

As shown in step S308, weight computation module 210 may calculate or estimate fetal weight 210A by a weighted sum of the base pixel/voxel weights throughout the fetus pixels/voxels as captured in volumetric scan 20.

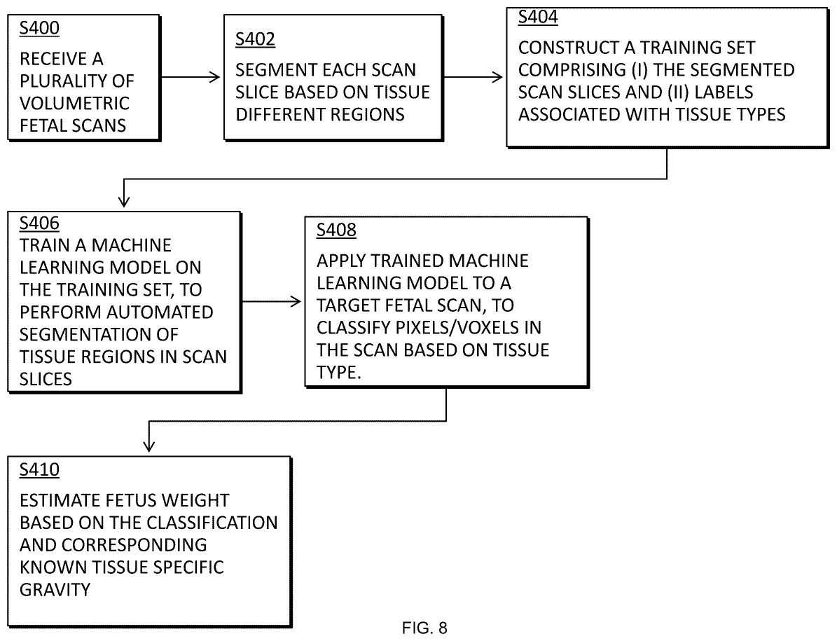

Reference is now made to FIG. 8, which is a flow diagram depicting a method of training an ML model (e.g., 115/125) to classify tissue types in fetal volumetric scans, according to some embodiments of the present invention. In some embodiments, fetal weight 210A may be calculated based, at least in part, on the classification.

According to some embodiments, and as shown in step S400, scan processing module 100 may receive a plurality of volumetric fetal scans 20, each including a sequence of 2D scan slices 20A. In some embodiments, image registration of various sequences (MR contrast) may be performed, and segmentation based on multi-parameters may be performed herein.

As shown in step S402, system 10 may annotate each scan slice 20A based on, e.g., tissue segments in each slice. For example, slice segmentation module 120 may initially segment each scan slice 20A, to indicate regions or segments associated with one of a set of tissue types (e.g., bone tissue, muscle tissue, adipose tissue, fluids, and the like). In some embodiments, scan slice annotation may be performed manually, using, e.g., expert annotators. Additionally, or alternatively, automated and/or semi-automated techniques may be used.

Additionally, or alternatively, slice segmentation module 120 may perform an initial segmentation of slices 20A into segments 120A, to segment an object and/or structure of interest in the scan, such as a fetus (e.g., fetal body, fetal brain, other fetal organs), and/or placenta. Additionally, or alternatively, slice segmentation module 120 may perform an initial segmentation of slices 20A into segments 120A that represent regions of different tissue types. In some embodiments, the initial segmentation may be performed using, e.g., a trained global segmentation algorithm. In some embodiments, a trained global neural network may be applied to perform the initial segmentation of the structures of interest. In some embodiments, the initial segmentation produces a classification of voxels in the scan as belonging to one of a set of classes.

As shown in step S404, in some embodiments, a training set may be constructed. This training set may include the annotated scans and labels associated with tissue types.

As shown in step S406, VOI module 110 may be, or may include an ML model 115, that may be trained on the training set to classify each pixel/voxel of scan 20 into one of the classes.

As shown in step S408, the trained ML model 115 may be applied to a target fetal scan, to classify each pixel/voxel of scan 20 to one of the classes. In other words, the trained ML model 115 may associate one or more (e.g., each) pixel/voxel of scan 20 with a respective tissue type.

As shown in step S410, weight computation module 210 may calculate or estimate fetal weight 210A based on the classification and corresponding known tissue specific gravity.

As explained herein, physicians and radiologists often resort to guessing which side of a presented scan slice image depicts the right side of the subject, and which represents the left side. It may be appreciated that the lateral information may indeed exist in the scan 20, but a physician will need to traverse through the sequence of slices 20A, and recognize specific structures depicted in multiple slices, in order to ascertain the correct orientation of a specific slice 20A of interest.

It may be appreciated that terms of directions (e.g., “above”, “below”, “left”, “right”, etc.) may be used in this context to indicate relative positions of anatomical structures, as normally displayed by a human subject standing upright (e.g., head being above toes, etc.).

According to some embodiments, annotation module 400 of FIG. 3 may facilitate automatic annotation or labeling of one or more brain structures in MRI scan 20 of a human subject. Such annotation or labeling may apply for one or more (e.g., each) slices 20A of scan 20, an indication of direction or orientation of structures depicted in that slice 20A.

For example, annotation module 400 may enable system 10 to receive a slice 20A depicting organs or structures such as brain hemispheres in a transverse plane of a fetus, and label or indicate the structures (e.g., the hemispheres) as pertaining to the left side, or the right side of the fetus.

According to some embodiments, annotation module 400 may automatically gain the orientation information based on a known relative positioning of at least three identified anatomic locations 400A.

Pertaining to the example of a transverse plane scan of a fetal brain, a first slice may depict a section of the cerebral cortex, without the cerebellum, and a second slice may include the cerebellum. According to anatomic knowledge, the cerebral cortex is superior to, or located above the cerebellum. Therefore, based on this anatomic knowledge, the relative positioning of two or more first points in the first slice (e.g., in the cerebral cortex) in relation to two or more second points in the second slice (e.g., in the cerebellum) is also known (the former being superior to the latter). Thus, a direction of the scan sequence may be determined, in a sense that the first slice 20A is determined to pertain to a plane that is superior to, or above a plane of the second scan. Based on this direction of the scan sequence, annotation module 400 may automatically ascertain whether a specific slice (e.g., the first slice) is presented as being viewed from above the scanned plane, or below it, and may thus ascertain which side of the presented image pertains to the right side of the subject, and which to the left.

In other words, annotation module 400 may receive a first slice 20A of the sequence of slices that was taken from a scan of a human subject or a fetus, and is included in VOI 110A. Annotation module 400 may also receive at least one second slice 20A of the sequence of slices, that is also included in VOI 110A.

Annotation module 400 may identify at least one first anatomic location 400A in the first slice, within VOI 110A, and may identify at least two second anatomic locations 400A in the at least one second slice, within VOI 110A.

As explained by the example above, based on anatomic knowledge of a relative positioning of the identified anatomic locations 400A (e.g., the at least one first anatomic location, and at least two second anatomic locations 400A), annotation module 400 may produce an annotation or label 400B for at least one brain structure, depicted in a slice of VOI 110A.

According to some embodiments, annotation module 400 may collaborate with slice segmentation module 120 to receive one or more segments 120A of identified structures depicted in slices 20A, and may use the received segments 120A as the identified anatomic locations 400A.

In other words, as elaborated herein, slice segmentation module 120 may apply an ML model 125 on the first slice and on the at least one second slice. As explained herein, the slice segmentation 120 ML model 125 may be trained to segment slices 20 into segments 120A, where each segment 120A is labeled as, associated with, or identified as pertaining to a specific structure depicted in VOI 110A. Slice segmentation 120 ML model 125 may thus segment each slice of the at least one first slice 20A and the at least one second slice 20A to a plurality of segments 120A that are identified as pertaining to specific structures and/or tissue types depicted in VOI 110A. Annotation module 400 may receive the segments 120A of the at least one first slice 20A and the at least one second slice 20A (alongside their respective identification of structure and/or tissue type). Annotation module 400 may identify the required anatomic locations 400A by identifying presence (or lack thereof) of specific segments 120A of the plurality of segments 120A as representing specific brain structures or tissue types of a predefined set of brain structures.

Pertaining to the example above, of a transverse plane scan of a fetal brain: In the first slice, annotation module 400 may identify two or more anatomic locations 400A as appearance of segments 120A representing specific brain structures, e.g., right and left hemispheres of the cerebral cortex, and a lack of appearance of a segment 120A representing another specific brain structure, e.g., a cerebellum or brain stem. In the second slice, annotation module 400 may identify at least one anatomic location 400A as appearance of a segment 120A representing a specific brain structure, e.g., the cerebellum.

Based on anatomical knowledge, annotation module 400 may deduce that the first slice correspond to a plane that is superior to, or above the plane of the second scan. In other words, based on the identification of segments 120A as pertaining to anatomical structures and/or tissue types, annotation module 400 may determine a direction of the sequence of slices 20A. In this example, the direction may be manifested as the first slice 20A being above the second slice 20A.

As explained herein, based on the determined direction, annotation module 400 may ascertain the lateral information, e.g., which hemisphere is the right hemisphere, and which is the left one. Annotation module 400 may subsequently annotate or label 400B the at least one brain structure by applying a label 400B to the one or more segments 120A. Label 400B may represent pertinence of a segment to a specific tissue, a specific organ, and/or a specific structure in slice 20A. Additionally, or alternatively, label 400B may represent pertinence of the relevant segment to a left-side brain structure or a right-side brain structure, based on the determined direction.

For example, label 400B may include labels such as “right hemisphere”, “left hemisphere”, “right lateral ventricle”, “left lateral ventricle”, “cerebellum”, “right eye”, “left eye”, etc.

As elaborated herein, annotation module 400 may identify the direction of scan sequence 20 by identifying three or more anatomic locations 400A. In the non-limiting example provided above, these anatomic locations 400A may be points or voxels included in (a) a right hemisphere, (b) a left hemisphere, and (c) a cerebellum. It may be appreciated that embodiments of the invention may use any set of three or more anatomic locations 400A, for which the positioning relation is anatomically known. For example, anatomic locations 400A may be points or voxels included in (a) a right eye, (b) a left eye, and (c) a brain stem. Other combinations of anatomic locations 400A may also be possible.

According to some embodiments, module 100 may include a landmark module 140, adapted to identify one or more landmarks 140A (also referred to herein as “anatomical landmarks 140A) in VOI 110A. In some embodiments, landmark module 140 may be, or may include at least one ML-based model 145, such as a CNN model. The landmark module 140 ML model 145 may be trained on a training dataset of expert-annotated slices to identify the one or more landmarks 140A. Landmarks 140A may include points or regions in a slice 20A, that depict geometrical or morphological features in the scanned subject. Additionally, or alternatively, landmarks 140A may include points or regions in a slice 20A, that depict specific positions in anatomical structures or organs of the scanned subject.

For example, landmarks 140A may include points or regions in a slice 20A that depict specific brain gyri, specific brain sulci, specific portions of brain lobes and the like.

According to some embodiments, landmark module 140 may identify three or more landmarks 140A in the brain, as depicted in slices of VOI 110A. Annotation module 400 may receive the three or more landmarks 140A as anatomic locations 400A for ascertaining a direction of the sequence of slices 20A based on anatomic locations 400A (e.g., the identification of three or more landmarks 140A), as elaborated herein.

Annotation module 400 may subsequently proceed to produce one or more label or annotation 400B based on the anatomic locations 400A (e.g., the landmarks 140A), as elaborated herein.

As known in the art, clinicians normally perform measurement of cranial and/or ocular distances in a process that is iterative, time consuming, repetitive, and prone to be inaccurate due to fatigue. For example, Cerebral Biparietal Diameter (CBD), Bone Biparietal Diameter (BBD) and Trans Cerebellum Diameter (TCD) distance measurements are normally manually performed by clinicians following established guidelines. These guidelines specify how to establish the scanning imaging plane, how to select the reference slice in a scan for each measurement, and how to identify anatomical landmarks for the required measurement. The CBD and BBD measurements are typically manually performed on the same identified slice based on the anatomical landmarks, and are drawn perpendicular to a manually-identified mid-sagittal line. The TCD is manually measured on a different reference slice by selecting the two antipodal landmark points on the fetal brain cerebellum contour, resulting in the cerebellum diameter. To perform these measurements, the clinician has to manually locate the fetal brain VOI in the scan, manually select an appropriate slice upon which measurements may be conducted (or rescan the fetus, in case no such slice is found), manually mark the required landmarks and MSL, and manually perform the desired measurement.

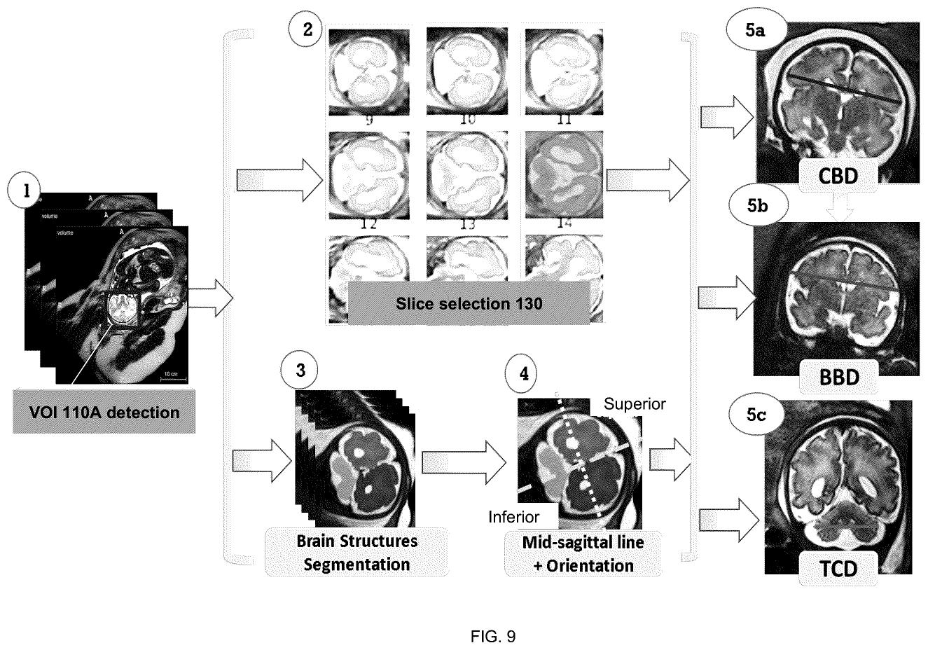

Reference is now made to FIG. 9, which is a schematic diagram depicting a fully automated method of performing cerebral fetal MRI biometric measurements, according to some embodiments of the invention.

As elaborated herein, and denoted by label (1) in FIG. 9 VOI module 110 may be, or may include an ML-based model (e.g., an anisotropic 3D U-Net classifier), trained to extract a VOI 110A from a fetal MRI scan 20.

Additionally, as elaborated herein and denoted by label (2) in FIG. 9, system 10 may include a slice selection module 130, that may be, or may include an ML-based model (e.g., a custom CNN model) 135. ML-based model 135 may be trained to select a reference slice 130A, upon which a desired measurement is to be performed. It may be appreciated that reference slice 130A may be selected from a plurality of slices 20A, pertaining to one or more scans 20.

As elaborated herein, slice segmentation module 120 may be, or may include an ML-based model (e.g., a multiclass U-Net classifier), trained to segment the one or more slices 20A into segments 120A representing anatomical structures. This is denoted by label (3) in FIG. 9, where a cerebellum is depicted as a green segment 120A, a left hemisphere is depicted as a blue segment 120A, and a right hemisphere is depicted as a red segment 120A.

As elaborated herein, system 10 may include a geometric algorithm module 222 (or “geometric module 222”, for short), adapted to compute the fetal brain midsagittal line (MSL) and fetal brain orientation vector 222C. This is denoted by label (4) in FIG. 9, where the MSL is depicted by a yellow line, and the and brain orientation vector 222C is depicted as a light blue line.

As elaborated herein, geometric module 222 may be further configured to compute a distance 220A based on one or more of the structure segments 120A, MSL and orientation vector. This is denoted by labels (5a-5c) in FIG. 9, where label 5a depicts a CBD distance, label 5b depicts a BBD distance, and label 5c depicts a TCD distance.

As elaborated herein, scan processing module 100 may receive an MRI scan 20 of a fetus, including at least one sequence of slices 20A. Scan processing module 100 may employ VOI module 110 to detect a VOI 110A representing a location of a brain of the fetus depicted in scan 20.

According to some embodiments, scan processing module 100 apply at least one ML model 145 on VOI 110A, to identify two or more landmarks 140A depicted in the scan.

For example, as elaborated herein, landmarks module 140 may be, or may include an ML model 145 such as a CNN model, trained to identify at least one landmark 140A from VOI 110A.

In another example, as elaborated herein, slice segmentation module 120 may be, or may include an ML model 125 such as a CNN model, trained segment slices 20A into segments 120A, such that each segment 120A may represent a specific structure or tissue depicted in the slice 20A. Embodiments of the invention may then apply a geometric algorithm (denoted in FIG. 3 as 222, 232) to segments 120A of at least one slice 20A, to identify at least one landmark (denoted in FIG. 3 as 222A, 232A), as elaborated herein. Landmarks 222A/232A are also referred to herein as “anatomical landmarks” 222A/232A, respectively.

According to some embodiments, fetal features module 200 may include a cranial measurements module 220, adapted to receive two or more landmarks depicted in scan 20. These two or more landmarks may be landmarks 140A (from landmarks module 140) and/or landmarks 222A (from geometric module 222). Cranial measurements module 220 may then automatically calculate or measure at least one cranial distance value 220A between the two or more landmarks (140A and/or 222A). The at least one cranial distance value 220A (or “distance 220A” for short) may, for example include a Cerebral Biparietal Diameter (CBD), a Bone Biparietal Diameter (BBD), a Trans-Cerebellum Diameter (TCD), a Front Occipital Diameter (FOD), a Vermian Height (VH), and Lateral Ventricle Width.

Additionally, or alternatively, fetal features module 200 may include an ocular measurements module 230, adapted to receive two or more landmarks depicted in scan 20. These two or more landmarks may be landmarks 140A (from landmarks module 140) and/or landmarks 232A (from geometric algorithm module 232). Ocular measurements module 230 may then calculate or measure at least one ocular distance value 230A between the two or more landmarks (140A and/or 232A). The at least one ocular distance value 230A (or “distance 230A” for short) may, for example include a Binocular Distance (BOD), an Interocular Distance (IOD), an Ocular Distance (OD), and a Lens Aligned Distance (OD-LA-OD).

According to some embodiments, fetal features module 200 may emit distance 220A/230A as a fetal feature 200A, and may include distance 220A/230A as part of a report 40 (e.g., as an evaluation 40A of a condition of the fetus).

Additionally, or alternatively, fetal features module 200 may transmit distance 220A/230A to condition assessment module 500, which may in turn analyze distance 220A/230A to perform a prediction of a condition of the fetus, based on the calculated at least one distance value.

For example, condition assessment module 500 may receive an ocular distance value 230A such as an IOD distance value 230A, and may compare the received distance value 230A (e.g., IOD) to a predetermined threshold value. According to some embodiment, the predetermined threshold value may be determined according to background medical data (e.g., GA) of the fetus and/or mother. Based on the comparison, condition assessment module 500 may evaluate 40A a condition of the fetus. For example, the inventors have experimentally shown that system 10 may successfully diagnose or predict a condition of hypotelorism in fetuses with a small IOD distance value 230A.

Additionally, or alternatively, condition assessment module 500 may calculate one or more normalized ocular distance metrics 230A, and use rule-based prediction to determine or predict a condition of the fetus based on the normalized ocular distance metrics 230A.

For example, condition assessment module 500 may calculate a normalized IOD distance 230A according to equation Eq. 1, below:

Normalized_IOD=IOD/(OD_{right}+OD_{left}) Eq. 1
where Normalized_IOD is the normalized IOD distance 230A, IOD is the IOD distance 230A calculated by ocular features module 230, and OD_{right}, OD_{left} are the OD distances 230A obtained for the right eye and left eye, respectively.

Condition assessment module 500 may subsequently predict or evaluate a condition of the fetus (e.g., hypotelorism, hypertelorism) based on normalized IOD distance 230A (e.g., if normalized IOD distance 230A falls beyond a predefined normal range).

In another example, condition assessment module 500 may calculate a normalized BOD distance 230A according to equation Eq. 2, below:

Normalized_BOD=BOD/(OD_{right}+OD_{left}) Eq. 2
where Normalized_BOD is the normalized BOD distance 230A,
BOD is the BOD distance 230A calculated by ocular features module 230,
and OD_{right}, OD_{left} are the OD distances 230A obtained for the right eye and left eye, respectively.

Condition assessment module 500 may subsequently predict or evaluate a condition of the fetus (e.g., hypotelorism, hypertelorism) based on normalized BOD distance 230A (e.g., if normalized BOD distance 230A falls beyond a predefined normal range).

It may be appreciated that the normalization of IOD distance 230A and BOD distance 230A may cause the normalized versions of these parameters to be agnostic to characteristics such as GA, gender, ethnicity, and the like, and may thus be used as robust indications 40A or predictors 40B of a variety of fetal conditions.

A common problem occurring in fetal MRI is caused by unpredictable and substantial fetal motion causing image artifacts, which subsequently limits clinical diagnosis based on image contrasts. Mitigation of motion artifacts is usually performed by fast, single-shot MRI and various retrospective motion corrections. Alternatively, multiple repetition of a scan in all relevant planes may be performed, and a manual selection may be applied to select a candidate scan for evaluation.

Embodiments of the invention may include an improvement over currently available methods of fetal MRI analysis by providing a process of automated selection of an optimal scan from a set of fetal MRI scans, and for classifying scan slices in the selected scan based on a probabilistic classification model.

In some embodiments, the process of the present invention may be performed offline, on a series of scans performed with respect to a fetus.

Additionally, or alternatively, the process of the present invention may be performed in real time or near-real time, with respect to newly completed scans, to determine scan quality and suitability for medical assessment.

According to some embodiments, module 100 may include a slice selection module 130, adapted to select at least one slice 20A of scan 20 as a reference slice, as elaborated herein.

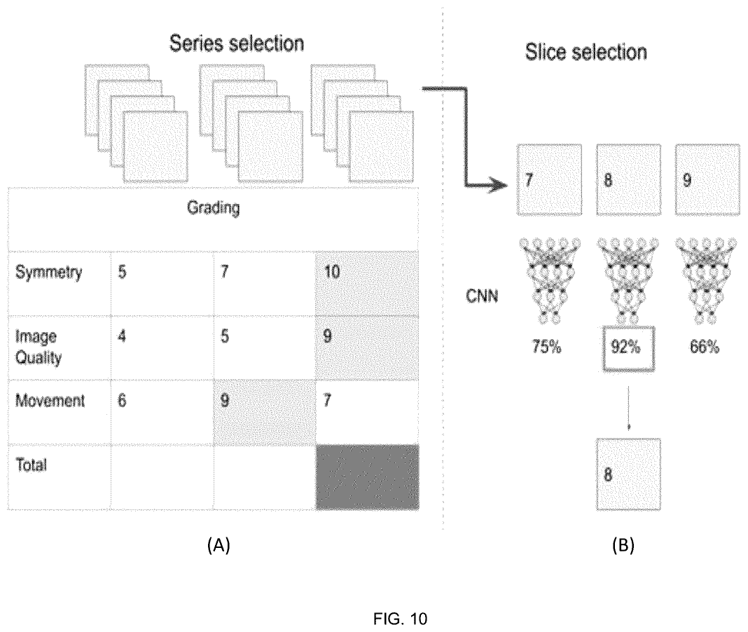

Reference is now made to FIG. 10, which is a schematic diagram depicting a process of scan slice selection, according to some embodiments of the invention.

According to some embodiments, system 10 may receive a plurality of scan sequences 20 (also referred to herein as a “scan series” 20). Panel A of FIG. 10 depicts an example of a first stage of reference slice selection, in which slice selection module 130 may select a specific scan sequence 20.

In the example depicted in panel A, each scan sequence may be graded according to one or more selection criteria.

For example, a criterion for scan 20 selection may be a criterion of symmetry. For example, slice selection module 130 may (a) calculate a metric of symmetry for one or more (e.g., each) slice 20A of the relevant scan 20, and (b) assign a symmetry score to the relevant scan 20, as a function (e.g., sum) of the individual slice 20A metrics of symmetry.

In another example, a criterion for scan 20 selection may be a criterion of image quality. For example, slice selection module 130 may (a) calculate a metric of image quality (e.g., sharpness, contrast, brightness, etc.) for one or more (e.g., each) slice 20A of the relevant scan 20, and (b) assign an image quality score to the relevant scan 20, as a function (e.g., sum) of the individual slice 20A metrics of image quality.

In yet another example, a criterion for scan 20 selection may be a criterion of movement. For example, slice selection module 130 may (a) calculate a metric of movement (e.g., within slices 20A, between slices 20A etc.) for one or more (e.g., each) slice 20A of the relevant scan 20, and (b) assign a movement score to the relevant scan 20, as a function (e.g., sum) of the individual slice 20A metrics of movement.

As depicted in panel A of FIG. 10, slice selection module 130 may subsequently aggregate the scores (e.g., symmetry score, image quality score, movement score), and select the highest scoring scan sequence 20 or series.

According to some embodiments, slice selection module 130 may include at least one ML model 135, such as a CNN-based classifier, that may be trained to produce at least one slice-specific score, as elaborated herein. Slice selection module 130 may apply ML model 135 on one or more (e.g., each) slices 20A of the highest scoring scan sequence 20, to produce one or more respective slice scores 135A. Slice selection module 130 may then select a reference slice 130A from the one or more slices of the selected scan 20, based on the respective slice scores.

In some embodiments, trained ML model 135 may be configured to classify scan slices 20A with respect to a specified type of desired medical use (e.g., a specified physical measurement).

According to some embodiments, ML model 135 may be trained to produce the slice score 135A for each specific slice 20A, where slice score 135A is a prediction of a probability of selection of the relevant slice by an expert (e.g., a human expert, such as a radiologist), for the purpose of measuring a specific distance 220A type (e.g., CBD/BBD or TCD).

Additionally, or alternatively, slice selection module 130 may include a plurality of ML models 135, each trained to produce slice-specific scores 135A as a prediction of a probability of selection of the relevant slice by the expert, for the purpose of measuring a respective plurality of distance 220A/230A types.

According to some embodiments, reference slice selection ML model 135 may include two separate ML models: one for CBD/BBD measurement reference slices 130A, and one for TCD measurement reference slices 130A. The one or more ML models 135 may be trained to perform reference slice selection 130A by using an annotated dataset that includes annotated selection of reference slices, and/or scoring of slices.

For example, during a training phase, the one or more ML models 135 may receive a training dataset that includes a plurality of manually (e.g., by a human expert) annotated slices 20A. The slices may be annotated in a sense that each annotation may associate a specific slice with a score and/or a label, indicating whether the slice is appropriate for performing a specific type of measurement (e.g., CBD/BBD or TCD). In a subsequent inference phase, the one or more ML models 135 may produce, for each target slice 20A a score 135A, representing a probability of selection of the relevant slice by an expert, for the purpose of performing the relevant measurement (e.g., CBD/BBD or TCD).

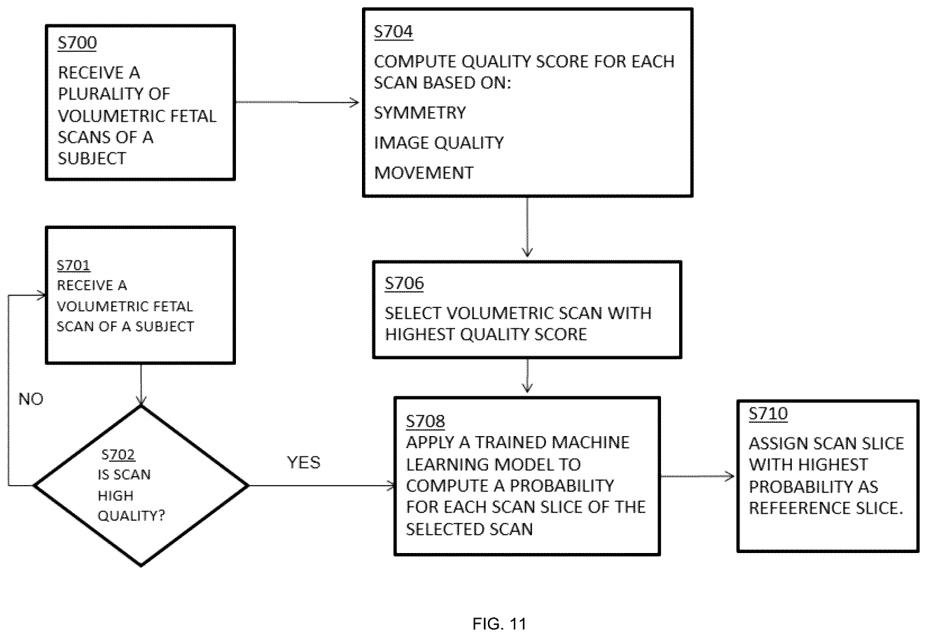

Reference is now made to FIG. 11, which is a flow diagram depicting an example of a method of selecting an optimal volumetric scan 20 and/or a volumetric scan slice 20A, according to embodiments of the invention.

In some embodiments, at step S700, the present method provides for receiving and preprocessing a plurality of volumetric scans 20 such as MRI scans, each may include a series of consecutive 2D slices or images 20A of a subject.

In some embodiments, at step S704, a quality score is computed for each scan 20 based on one or more of: symmetry, image quality, and movement. For example, a fetus may move during an MRI scan, resulting in a loss of image detail and motion artefacts which would result in a low movement score.

At step S706, at least one volumetric scan 20 with the highest quality score may be selected.

Additionally, or alternatively, in order to reduce costs of redundant volumetric scans, at step S701, one volumetric fetal scan 20 of a subject is received.

Following S701, at step S702 the received volumetric scan 20 may be assessed for quality as in step S704. If the scan quality is above a predetermined threshold, then the scan may be used in step S708. In a complementary manner, if the scan quality is below the predetermined threshold, then another scan may be requested for assessment. For example, as depicted in FIG. 2 system 10 may automatically communicate with scanning device 50 (e.g., an MRI scanner) to reissue the scan. Additionally, or alternatively, system 10 may produce a notification to a user, via a user interface (e.g., output 8 of FIG. 1), and prompt the user to manually perform the scan 20.

In some embodiments, at step S708 a trained ML model (e.g., ML model 135) may be applied to the selected scan to compute a probability score 135A for each scan slice. The higher the probability score 135, the more likely a scan slice 20A is to be the optimal slice to be used as a reference slice 130A.

At step S710, a scan slice 20A with the highest probability may be assigned as a reference slice 130A and outputted to the user.

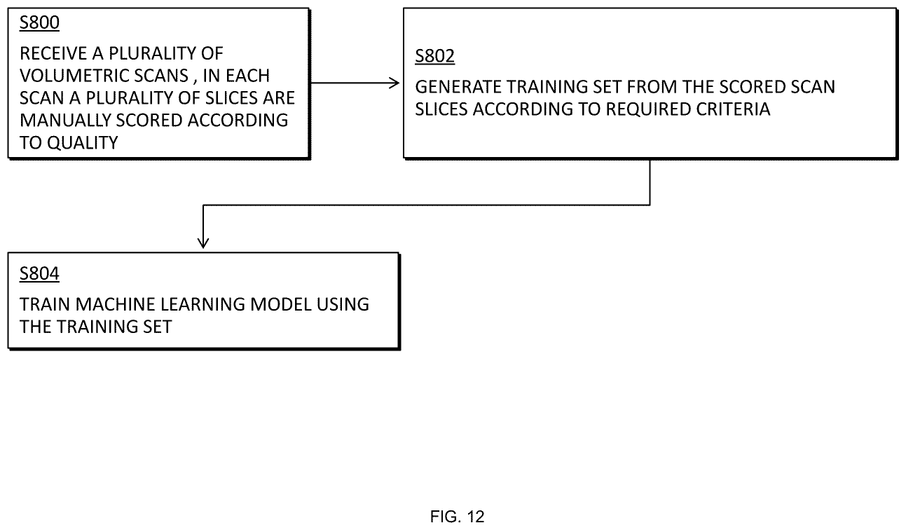

Reference is now made to FIG. 12, which is a flow diagram depicting an example of a method of training an ML model (e.g., ML model 135) to optimally select volumetric scan slices, according to some embodiments of the present invention.

At step S800, a plurality of volumetric scans is received, each comprising a series of 2D slices, wherein a plurality of slices of each scan are scored (e.g., manually examined and scored) according to quality.

At step S802, a training set is generated from the scored scan slices according to required criteria. In some embodiments, a training set of the present invention may include a plurality of labeled or annotated slice scans 20A. In some embodiments, one or more training sets may be created, e.g., to train corresponding models with respect to each one or more of the desired measurements. For example, one training set may be used to train an ML model (e.g., slice selection ML model 135) to select a reference slice on which CBD and/or BBD measurements are performed, and a different training set may be used to train another ML model 135 to select a reference slice 130A on which TCD measurement is performed.

In some embodiments, at step S804, a trained ML model (e.g., slice selection ML model 135) may be configured to select a reference slice 130A in a scan 20, given a desired measurement (e.g., CBD, BBD, TCD, and the like) to be performed with respect to he selected scan 20.

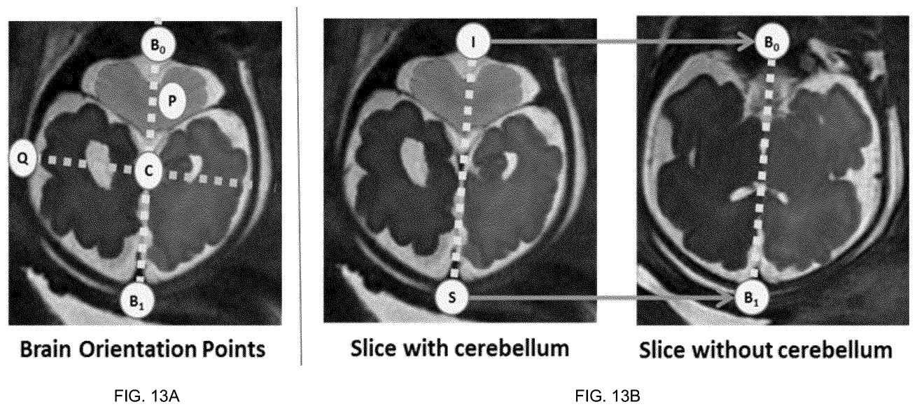

Reference is now made to FIGS. 13A and 13B, which are images depicting steps in a method of performing cerebral fetal MRI biometric measurements, according to some embodiments of the invention.

According to some embodiments, system 10 may apply the ML model 125 of segmentation module 120 on a subset of the sequence of slices 20A, that includes the reference slice 130A, to perform multi-class segmentation of one or more (e.g., each) slice to a plurality of segments 120A. Segmentation module 120 may thus identify each of the segments as representing a brain structure of a predefined set of brain structures. The predefined set of brain structures may include, for example a right hemisphere, a left hemisphere, and a cerebellum.

For example, as shown in FIGS. 13A and 13B, a cerebellum is depicted as a green segment 120A, a left hemisphere is depicted as a blue segment 120A, and a right hemisphere is depicted as a red segment 120A.

According to some embodiments, geometric module 222 may be configured to calculate a mid-sagittal line (MSL) based on the fetal brain multi-class structure segmentation. In FIGS. 13A and 13B the MSL 222B is depicted as a yellow, dashed line, indicated between points B0 and B1; the two intersection points of the MSL 222B and the fetal brain VOI 110A.

Geometric module 222 may compute the MSL 222B as the minimal margin line that separates the left and right fetal cerebral hemispheres. According to some embodiments, geometric module 222 may compute the MSL 222B using a Support Vector Machine (SVM) classifier with a linear kernel, according to equation Eq. 3, below:

$$[ \frac{1}{n} {\sum }_{i = 1}^{n} max ( 0 , 1 - {γ}_{i} \cdot ( w \cdot {x}_{i} - b ) ) ] + λ {‖ w ‖}^{2} \qquad Eq . 3$$

where xᵢ is the pixel coordinates vector,
w=(w₀, w₁) is the linear kernel weights vector,
yᵢ is the cerebral hemisphere index (−1 left, +1 right),
λ is a predefined regularization parameter, and
b is the bias weight.

In some embodiments, the SVM solution yields the values of w and b from which the mid-sagittal line equation is computed as:

$$γ = - \frac{{w}_{0}}{{w}_{1}} x - \frac{b}{{w}_{1}} .$$

According to some embodiments, geometric module 222 may be configured to evaluate reliability of calculation of the mid-sagittal line. For example, when mid-sagittal line angles

$$( e . g . , - \frac{{w}_{0}}{{w}_{1}} )$$

of adjacent slices sitter beyond a predefined threshold, geometric module 222 may produce a notification of an unreliable result. Additionally, or alternatively, geometric module 222 may reissue computation of MSL 222B in one or more slices. Additionally, or alternatively, geometric module 222 may communicate with scanner 50 to automatically reissue a new scan 20.

According to some embodiments, geometric module 222 may calculate a brain orientation vector 222C in one or more slices 20A, including the reference slice 130A, based on the multi-class segmentation 120A. Geometric module 222 may calculate the brain orientation vector 222C by applying geometric computations, and relying on the anatomical location of the cerebellum, which is inferior to the cerebral hemispheres, and located at the back of the skull.

As shown in FIG. 13B, the brain orientation vector 222C may connect between an inferior point (denoted ‘I’) and a superior point (denoted ‘S’) along the mid-sagittal line, thus showing the orientation of the fetus head (in this example, facing down).

As depicted in FIG. 13A and FIG. 13B, the mid-sagittal line may intersect the fetal brain VOI at points B0 and B1, from which geometric module 222 may calculate a midpoint or center of mass C. Geometric module 222 may a line QC that is normal to the MSL 222B, and intersects the fetal brain ROI at point Q. Geometric module 222 may then sample an arbitrary point P inside the cerebellum segment 120A, and classify point P with respect to the sign of the cross-product QC×CP, where QC is the vector connecting point Q to point C, and CP is the vector connecting point C to point P. Since the cerebellum is inferior to the brain hemispheres, all points whose cross-product sign is positive, correspond to the inferior part of the brain. In a complementary manner, all points whose cross-product sign is negative, correspond to the superior part of the brain. Geometric module 222 may thus determine the superior (S) and inferior (I) points. In other words, geometric module 222 may thus determine which of points B0 and B1 corresponds to the inferior (I) side of MSL 222B, and which corresponds to the superior (S) side of MSL 222B.

According to some embodiments, geometric module 222 may repeat this process on one or more (e.g., all) slices 20A that contain the cerebellum segment 120A. As depicted in FIG. 13B, geometric module 222 may then apply the orientation vector to one or more (e.g., all) slices without the cerebellum, by computing the Euclidean nearest neighbor distance in the slice plane.

In some embodiments, geometric module 222 may determine the reliability of the brain orientation vector 222C computation by computing the brain orientation vector 222C on randomly sampled points in the cerebellum in each slice that includes a cerebellum segment 120A.

In some embodiments, in case the computed brain orientation vectors 222C are not in agreement with that of the reference slice 130A, geometric module 222 may issue a brain orientation reliability warning. Additionally, or alternatively, geometric module 222 may reissue calculation of the brain orientation vector 222C and/or the MSL 222B.

Additionally, or alternatively, geometric module 222 may automatically communicate with scanning device 50 (e.g., MRI scanner) to reissue a new scan 20.

According to some embodiments, geometric module 222 may identify two or more landmarks 222A based on the midsagittal line and the brain orientation vector 222C, as elaborated herein.

In some embodiments, the method includes computing the CBD, BBD, and TCD measurements with a geometric method. Embodiments of the invention may measure, or calculate cranial distances 220A (e.g., CBD, BBD, and TCD) by applying respective geometric algorithms, as elaborated herein.

According to some embodiments, geometric algorithm module 222 may measure or compute CBD and BBD distances 220A on the same reference slice 130A, and may measure or compute TCD on a different reference slice 130A.

In some embodiments, geometric algorithm module 222 may measure CBD distance 222A by calculating the maximal width of the cerebral hemispheres superior to the Sylvian fissure, and perpendicular to the mid-sagittal line.

In some embodiments, geometric algorithm module 222 may measure BBD distance 222A by continuing the CBD line until it intersects an inner boundary of the fetal skull in both directions, and calculating the distance between the intersection points.

In some embodiments, geometric algorithm module 222 may measure TCD distance 222A by calculating a maximal diameter of the cerebellum.

In some embodiments, the inputs are the reference slice k and the list L(k) of adjacent candidate reference slices for each of the two reference slices and the measurements are computed on slice k and on all slices of L(k). In some embodiments, the resulting measures are then evaluated using a probability-based method, a maximum-based method or both to produce a single measurement value with the highest confidence level. In some embodiments, the probability-based method returns the measurement on the slice k (the slice with highest probability). In some embodiments, the maximum-based method returns the measurement on a slice in L (k) whose value is maxima. In some embodiments, the probability-based method is used for BBD measurements. In some embodiments, maximum-based method is used for TCD, CBD or both measurements.

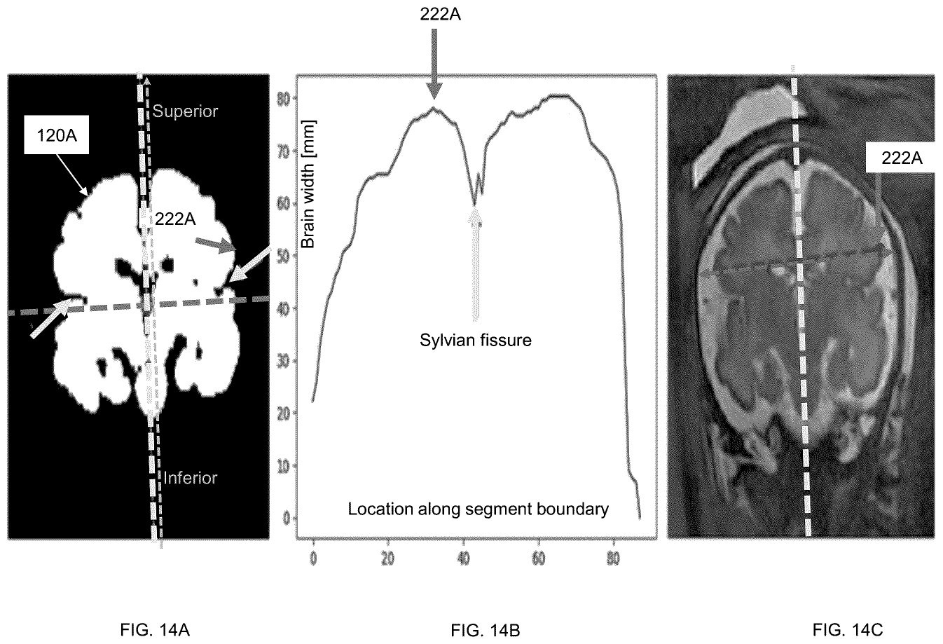

Reference is now made to FIGS. 14A, 14B and 14C, which are images depicting steps in a method of performing cerebral fetal Mill biometric measurements (e.g., CBD and BBD), according to some embodiments of the invention.

For example, as depicted in FIG. 14C, geometric module 222 may compute the CBD distance 220A measurement in the reference slice 130A, based on the MSL 222B (yellow dashed line in FIGS. 14A, 14C), the brain orientation vector 222C (orange dashed arrow in FIG. 14A) and brain structures' segmentation 120.

Geometric module 222 may compute the cerebrum width profile perpendicular to the MSL 222B, from the cerebral brain segment 120A boundary. This computation is demonstrated in FIG. 14B, which is a graph depicting the cerebrum width profile when traversing along the cerebral brain segmentation 120A boundary.

Geometric module 222 may then identify a first anatomic landmark 222A that is the Sylvian fissure location (yellow arrows in FIGS. 14A and 14B). Geometric module 222 may compute the location of the Sylvian fissure anatomic landmark 222A by finding the local minimum of width profile that is the closest to, and superior to the mass center of mass of the cerebral brain segment 120A (red dot in FIG. 14A) in the MSL 222B.

Geometric module 222 may then identify a second anatomic landmark 222A that is the CBD measurement point (blue arrow in FIGS. 14A and 14B). The CBD measurement point anatomic landmark 222A may be the maximal width of the cerebral hemispheres, directly superior to the Sylvian fissure (as defined by the orientation vector), and perpendicular to the MSL 222B.

Geometric module 222 may then calculate the CBD distance value 220A as the total width of the cerebrum segment 120A, from the CBD measurement point anatomic landmark 222A, and perpendicular to the MSL 222B. The CBD distance is depicted as a red dashed line in FIG. 14C.

In another example, as also depicted in FIG. 14C, geometric module 222 may compute the BBD distance 220A measurement in the reference slice 130A, based on the MSL 222B, the brain orientation vector 222C and brain structures' segmentation 120.

According to some embodiments, geometric module 222 may compute the BBD distance 220A (green arrow in FIG. 14C) by extending the CBD line (red line FIG. 14C) to the skull contour on the same reference slice 130A, to find two anatomic landmark 222A that are BBD measurement points, and subsequently calculating the distance 220A between these anatomic landmark 222A.

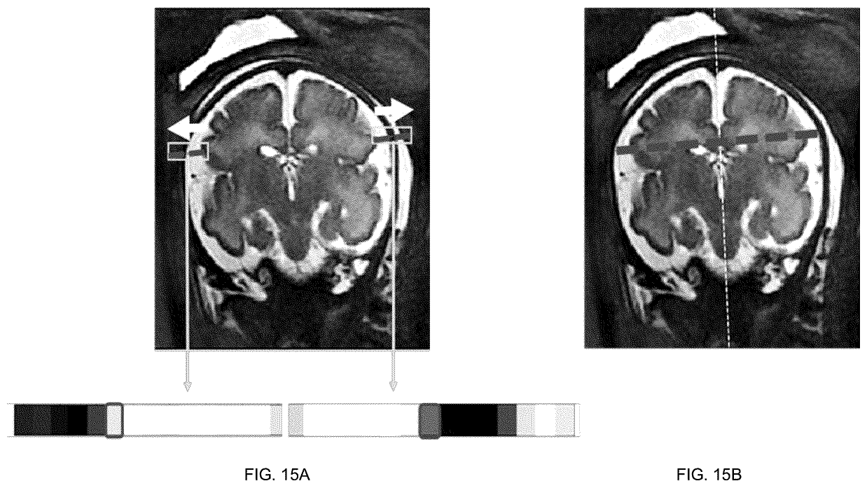

Reference is now made to FIGS. 15A and 15B, which are images depicting steps in a method of performing cerebral fetal MRI biometric measurements (e.g., BBD measurement), according to some embodiments of the invention. FIG. 15A shows an extension of the CBD measurement line towards the fetal skull (red line), superimposed on the original fetal MRI slice and intensity profile along the CBD line (below). The pixels chosen as the inner boundary mark the locations of the inner fetal skull boundary. FIG. 15B depicts the subsequent BBD measurement (red line) and mid-sagittal line (yellow).

It may be appreciated that there may be various methods to determine the exact anatomic landmark 222A points of BBD measurement. In some embodiments, and as depicted in FIG. 15A, geometric module 222 may find the BBD measurement landmarks 222A by computing the intensity derivative along the extended CBD line, and detecting one or more local maxima of the derivatives (e.g., corresponding to edges of the skull). Geometric module 222 may then identify the inner skull contour pixels by selecting a point that (a) has the maximal derivative value of the one or more local extrema, (b) is closest to the segmented cerebral brain boundary, and/or (c) has an intensity value that surpasses a predefined threshold. The threshold value may be selected so as to filter out MR scanning imaging artifacts on the CSF, which may appear as dark lines or spots, and therefore may cause noise when analyzing the intensity extrema. Geometric module 222 may relate to the selected inner skull contour pixels as the BBD measurement landmarks 222A, and perform a Euclidean distance calculation between the BBD measurement landmarks 222A, to obtain the required BBD distance 220A.

According to some embodiments, geometric module 222 may be configured to evaluate reliability of BBD measurements. For example, geometric module 222 may be configured to perform BBD measurement landmarks 222A on (a) a first, original version of reference slice 130A, and (b) on a second version of reference slice 130A, after applying contrast limited adaptive histogram equalization (CLAHE). The rationale for this approach is that the fetal cerebrospinal fluid (CSF) might yield intensity inhomogeneity and imaging artifacts, so enhancing and equalizing the contrast may enhance the borders between CSF and brain parenchyma. Geometric module 222 may then compare the BBD measurement landmarks 222A obtained from the two versions of reference slice 130A, and evaluate reliability of the selection of BBD measurement landmarks 222A based on the comparison. For example, a large Euclidean difference (e.g., beyond a predefined threshold) may indicate that the BBD measurement is unreliable. Geometric module 222 may subsequently produce a notification and/or initiate repetition of the scan and/or distance measurement process.

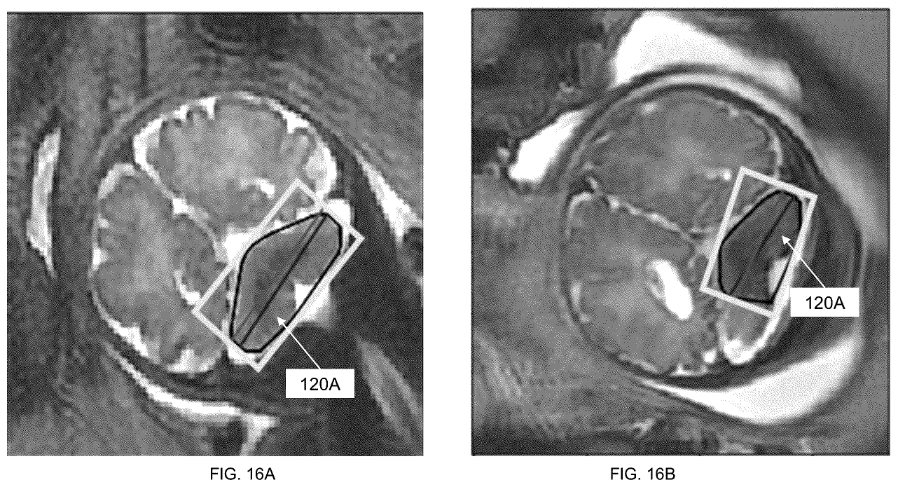

Reference is now made to FIGS. 16A and 16B, which are images depicting two fetal Mill biometric measurements (e.g., TCD measurements), as provided by embodiments of the invention.

In some embodiments, the input for the TCD measurement step is a reference slice 130A and a corresponding fetal brain structures segmentation 120A. In some embodiments, geometric module 222 may calculate the TCD distance as a measurement of the maximal diameter of a cerebellum contour convex hull. In some embodiments, cerebellum contour convex hull is computed using the QuickHull algorithm.

According to some embodiments, geometric module 222 may obtain (from slice selection module 130), a reference slice 130A for measuring the TCD distance 220A. Additionally, geometric module 222 may obtain (from segmentation module 120) a segment 120A, representing the cerebellum, as depicted in reference slice 130A.

As shown in FIGS. 16A, 16B, geometric module 222 may calculate a convex hull of the cerebellum segment 120A (depicted by a black contour). The term “convex hull” (also commonly referred to as a “convex envelope”) may be used herein to refer to the smallest convex set that contains a specific shape, in this case—the shape of the cerebellum segment 120A.

Based on the cerebellum convex hull, geometric module 222 may calculate a TCD line (depicted as a blue line), as a diameter of the convex hull. The term “diameter” may refer in this context to a maximal distance between any pair of points on the cerebellum convex hull.

Additionally, geometric module 222 may calculate a rectangular bounding box (depicted as a peach-color box), defining the cerebellum segment 120A, and further calculate a diameter or a length (depicted as a red line) of the longest axis of the bounding box.

In some embodiments, geometric module 222 may evaluate a reliability of the TCD measurement according to the bounding box long axis length (e.g., diameter): In some embodiments, if the angle between the TCD line (blue line) and the diameter of the bounding box (red line) does not exceed a predefined value (as depicted in FIG. 16A), then geometric module 222 may determine that the TCD measurement is reliable.

Alternatively, if the angle between the TCD (blue line) and the diameter of the bounding box exceeds a predefined value (as depicted in FIG. 16B), then geometric module 222 may determine that the TCD measurement is unreliable.

In instances where geometric module 222 determines that the TCD measurement is unreliable, geometric module 222 may subsequently issue a TCD measurement reliability warning. Additionally, or alternatively, geometric module 222 may issue recalculation of reference slice 130A. Additionally, or alternatively, geometric module 222 may automatically communicate with scanning device 50 (e.g., Mill scanner) to reissue a new scan 20 of the fetus.

In some embodiments, in the step or all steps within the present methods an automatic evaluation of reliability is performed. In some embodiments, in instances wherein a warning is issued in one or more steps/stages, a radiologist may inspect the result, make manual corrections as appropriate, or disregard the results. In some embodiments, reliability estimation facilitates the use of the proposed method in a clinical environment.

In some embodiments, computation reliability warnings are issued for: 1) unreliable reference slice selection, probability of the selected slice below a predefined threshold; 2) unreliable fetal brain structure segmentation and/or fetal brain orientation vector 222C, when brain orientation vectors 222C for random points sampled on the cerebellum differ; 3) unreliable mid-sagittal line, when the mid-sagittal line angles of adjacent slices differ; 4) unreliable BBD measurement when the measurements on the original and CLAHE-enhanced reference slices differ; 5) unreliable TCD measurement, when the line angles between two measurements of the fetal brain convex hull diameter and the cerebellum bounding box long axis differ.

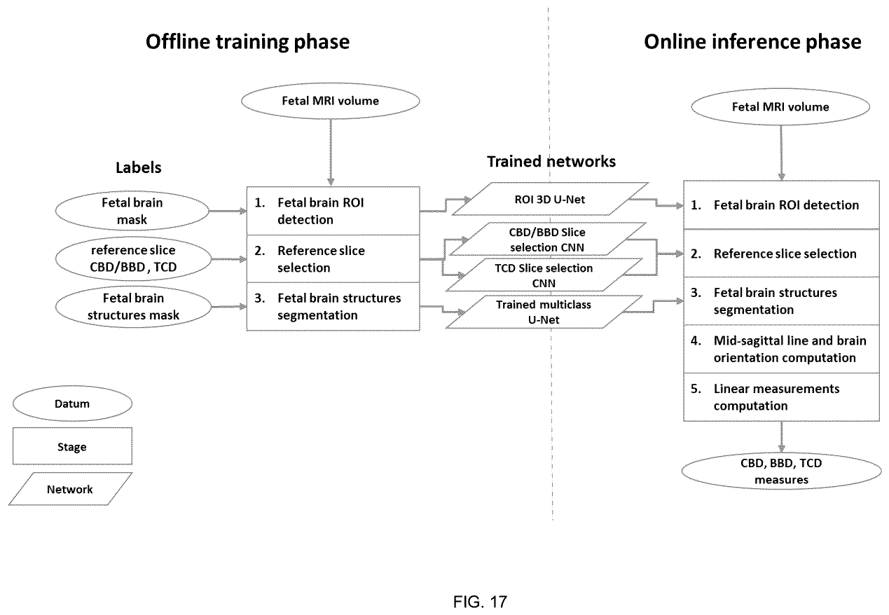

Reference is now made to FIG. 17, which is a flow diagram showing offline training (left) and online inference (right) phases of a method of performing cerebral fetal Mill biometric measurements, according to some embodiments of the invention.

As elaborated herein, embodiments of the invention may apply a multi-stage process for calculating biometric distances 220A/230A. In a first stage, VOI module 110 may use an ML model 115 such as an anisotropic 3D U-Net classifier to compute, or identify a volume of interest (VOI) of a fetal brain. In a second stage, slice selection module 130 may use an ML model 135 such as a convolutional neural network (CNN), to select at least one reference slice 130A. In a third stage, segmentation module 120 may use an ML model 125 such as a multi-class U-Net classifier to perform slice-wise fetal brain structure segmentation 120A. In a fourth stage, geometric algorithms module (e.g., 220) may compute the fetal brain MSL 222B and fetal brain orientation vector 222C, and in a fifth stage, geometric algorithms module may calculate biometric distances (e.g., CBD, BBD and TCD measurements).

As shown on the left panel of FIG. 17, embodiments of the invention may include an offline training phase, in which ML models 115, 125 and 135 may be trained individually on annotated training and/or validation datasets.

During The offline training phase, ML models 115, 125 and 135 may be trained according to labeled datasets, as denoted by the oval label elements.

ML model 115 may be trained to perform fetal brain VOI detection 110A by using an annotated dataset that includes fetal brain masks.

As elaborated herein, ML model 135 may be trained to perform reference slice selection 130A by using an annotated dataset that includes annotated selection of reference slices, and/or scoring of slices. According to some embodiments, reference slice selection ML model 135 may include two separate ML models: one for CBD/BBD measurement reference slices 130A, and one for TCD measurement reference slices 130A. Additionally, or alternatively, reference slice selection ML model 135 may be trained twice: once to select CBD/BBD reference slices 130A and once to select TCD measurement reference slices 130A.

As elaborated herein, ML model 125 may be trained to produce slice-wise fetal brain structure segmentation by using an annotated dataset that includes annotated (e.g., manually segmented) fetal brain, fetal head and/or fetal body slices 20A.

As shown by the parallelograms of FIG. 17, the outcome of the training phase may include three or more trained networks: one or more for fetal brain ROI detection 115, one or more (e.g., two) for reference slice selection 135, and one or more for fetal brain structure segmentation 125.

As shown on the right panel of FIG. 17, In a subsequent, online inference phase, the trained ML models 115, 125, 135 may be used for inference. During the inference phase, ML model 115 may produce fetal VOI detection 110A as elaborated herein; ML model 135 may select at least one reference slice 130A as elaborated herein; ML model 125 may segment one or more slices (including reference slice 130A) to segments 120A as elaborated herein; Geometrical module 222/232 may calculate at least one landmark 222A/232A (or receive one or more landmarks 140A from landmark ML 145). Additionally, or alternatively, geometrical module 222/232 may calculate at least one of an MSL 222B and/or an orientation vector 222C. Geometrical module 222/232 may subsequently calculate or measure one or more biometric distances 220A/230A based on at least one of the landmarks (140A/222A/232A), segments 120A, MSL 222B and/or orientation vector 222C, as elaborated herein.

In some embodiments, the present invention provides for classification of fetal tissue type in volumetric scan slices, by mapping corresponding pixel/voxel intensity values as captured in the volumetric scan slices to tissue types, based, e.g., on radio-density values associated with pixel/voxel intensity values.

According to another aspect of some embodiments of the present invention, there is provided a method for predicting fetal development and developmental disorders, birth weight, neonate outcome, based, at least in part, on determining a placental vascular network, integrity, volume, structure from volumetric scans.

It may be appreciated that fetal ocular biometrics are important parameters for fetal growth evaluation and detection of congenital abnormalities during pregnancy such as hypertelorism, hypotelorism, microphthalmia and anophthalmia, as they can be part of a genetic syndrome or may be related to a developmental abnormality. Accurate measurements can support improved diagnosis, pregnancy, and birth management. For example, ocular biometric distances 230A such as Binocular Distance (BOD), Interocular Distance (IOD), and Ocular Diameter (OD) are typically measured manually in routine clinical practice, and thus are dependent on the annotator's expertise.

According to some embodiments, system 10 be configured to perform additional types of distance measurements. For example, as shown in FIG. 3, fetal features module 200 may include an ocular measurements module 230, adapted to automatically perform measurement of ocular distances 230A, as elaborated herein.

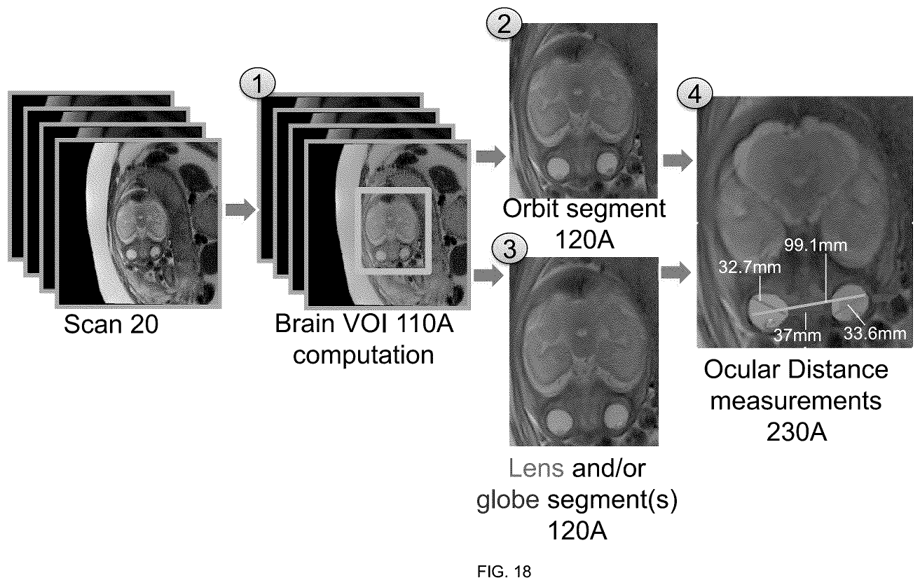

Reference is now made to FIG. 18, which is a schematic diagram depicting a method of performing ocular fetal Mill biometric measurements, according to some embodiments of the invention.

As shown in FIG. 18, system 10 may receive a scan 20 from a scanning device 50 such as an Mill scanner.

As shown in label (1) of FIG. 18, VOI module 110 may segment the fetal brain VOI 110A using a 3D two-stage anisotropic U-Net, as elaborated herein (e.g., in relation to FIG. 3). VOI module 110 may then crop VOI 110A using an axis-aligned 3D bounding box of the resulting segmentation.

As shown in labels (2) and (3) of FIG. 18, slice selection module 130 may select at least one reference slice 130A depicting fetal eye orbits, as elaborated herein (e.g., in relation to FIG. 3). Subsequently, segmentation module 120 may apply at least one ML-based model 135 to segment reference slice 130A to a plurality of segments 120A, each corresponding to a specific structure in the fetal head. For example, segmentation module 120 may apply at least one first ML model 135 to segment 120A fetal orbits, as depicted in label (2) of FIG. 18. Additionally, or alternatively, segmentation module 120 may apply at least one second ML model to segment 120A fetal lenses and/or globes, as depicted in label (2) of FIG. 18. It may be appreciated that the at least one first ML model 135 may, or may not be the same as the at least one second ML model 135.

According to some embodiments, during a training stage, the at least one ML model 135 may receive a training dataset that includes a plurality of manually annotated slices 20A of relevant segments 120A. The segments may be annotated in a sense that each annotation may associate a specific segment with an appropriate structure of the fetal body, head and/or brain. In this example, the annotated training dataset may include labels or annotations of ocular structures (e.g., orbits, globes, lenses, etc.). Additionally, or alternatively, the annotated training dataset may include labels or annotations of brain structures (e.g., left and right hemispheres, cerebellum, specific brain gyri, specific brain sulci, specific portions of brain lobes and the like).

As elaborated herein, ML model 135 may be a 2D U-Net CNN model, that includes an encoder portion (e.g., a 34-layer Residual Network (Resnet34) encoder) and a decoder portion. According to some embodiments, to overcome scarcity of annotated images, the encoder may be pre-trained based on a training dataset such as the ImageNet dataset, to obtain pre-trained weights. In addition, during the training stage, data augmentations such as 2D rotation, brightness adjustment, and contrast adjustment and the like may be applied to the training dataset.

According to some embodiments the at least one first ML model 135 may cluster the orbit segmentation 120A results to form a plurality of connected components, and may select the two largest connected components as correspond to the two fetus' eyes. Additionally, or alternatively, the at least one second ML model 135 may cluster the lens and globe segmentation 120A results to form a plurality of connected components model, and may select the two largest clusters, each one composed of lens and globe voxels, as corresponding to the fetus' globe and lens structures.

As shown in label (4) of FIG. 18, ocular measurements module 230 may automatically measure or calculate one or more ocular distances 230A based on ocular segments 120A (e.g., segments 120A of orbits, globes, lenses, and the like), as elaborated herein.

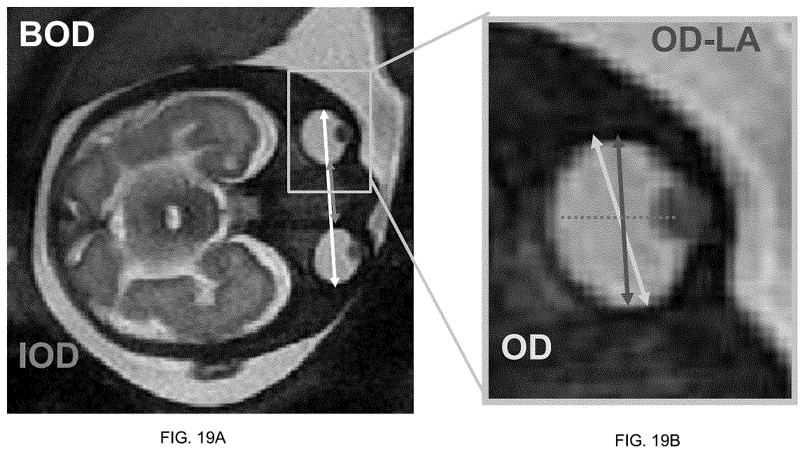

Reference is also made to FIGS. 19A and 19B, which are images depicting 2D fetal ocular measurements on a representative fetal MRI scan slice, as provided by embodiments of the invention.

According to some embodiments, ocular measurements module 230 may include a geometric algorithms module 232 (or “geometric module” 232 for short). Geometric module 232 may be configured to apply at least one geometric algorithm on the one or more ocular segments 120A, to calculate the one or more ocular distances 230A.

For example, geometric module 232 may calculate a 2D Binocular Distance (BOD) 230A (FIG. 19A, white arrow), as the maximum distance found between any two voxels between the two orbit segments 120A, on all slices 20A.

In another example, geometric module 232 may calculate a 2D Interocular Distance (IOD) 230A (FIG. 19A, blue arrow), as the minimum distance found between two voxels between the two orbit segments 120A, on all slices 20A.

In another example, geometric module 232 may calculate a 2D Ocular Diameter (OD) 230A (FIG. 19B, beige arrow), as the maximum distance between any two voxels within a single orbit segment 120A (e.g., for both eyes), on all slices 20A.

In another example, geometric module 232 may calculate a 2D OD-LA (Lens Aligned Ocular Diameter) distance as the maximum diameter of the globe boundary voxels, perpendicular to the line between globe center and lens center of the eye.

Additionally, or alternatively, geometric module 232 may calculate 3D BOD, IOD and/or OD distances in a similar manner as elaborated herein in relation to 2D measurements, but on the total orbit volume.

As elaborated herein, fetal features module may be configured to calculate a cranial distance 220A or ocular distance 230A based on two or more landmarks. These two or more landmarks may be landmarks 222A/232A, obtained from geometric algorithms' modules 222 and 232 respectively, or landmarks 140A, obtained from landmark module 140 (e.g., from ML model 145). According to some embodiments, landmark module 140 may further calculate reliability of landmarks 140A, using a Gaussian Mixture Model 146, as elaborated herein.

According to some embodiments, during an inference phase, landmark module 140 may receive at least one target slice 20A, apply an iterative process to produce a plurality of landmark candidates 140A′. In each iteration, landmark module 140 may employ ML model 145 to identify at least one landmark candidate 140A′ on the at least one target slice Landmark module 140 may then apply a spatial transform (e.g., rotation) on the at least one target slice 20A, to receive a transformed version of the at least one target slice 20A. In a subsequent iteration, ML model 145 may repeat the identification of landmark candidates 140A′ on the transformed version, to produce a new set of landmark candidates 140A′. This iterative process may continue until a stop condition is met (e.g., until a predefined number of iterations is performed).

Landmark module 140 may subsequently model the distribution of location of landmark candidates 140A′, e.g., by using a bi-modal (e.g., represent two clusters) Gaussian Mixture Model (GMM). Landmark module 140 may then calculate a Bayesian likelihood of the GMM model. The GMM Bayesian likelihood may manifest an estimate of landmark reliability: When the Bayesian likelihood value is low, the landmark locations are spatially dispersed, so their distribution may not be bi-modal. In this case, landmark module 140 may label the landmarks as unreliable and may issue a request (e.g., via output 8 of FIG. 1) to perform the labeling of landmarks manually (e.g., by an expert, via input 7 of FIG. 1). Additionally, or alternatively, landmark module 140 may communicate with scanner 50, to reissue the scan 20.

In a complementary manner, if the Bayesian likelihood value is high, then landmark module 140 may merge one or more landmark candidate 140A′ together (e.g., select a mean value of the corresponding GMM modality), to produce at least one landmark 140A.

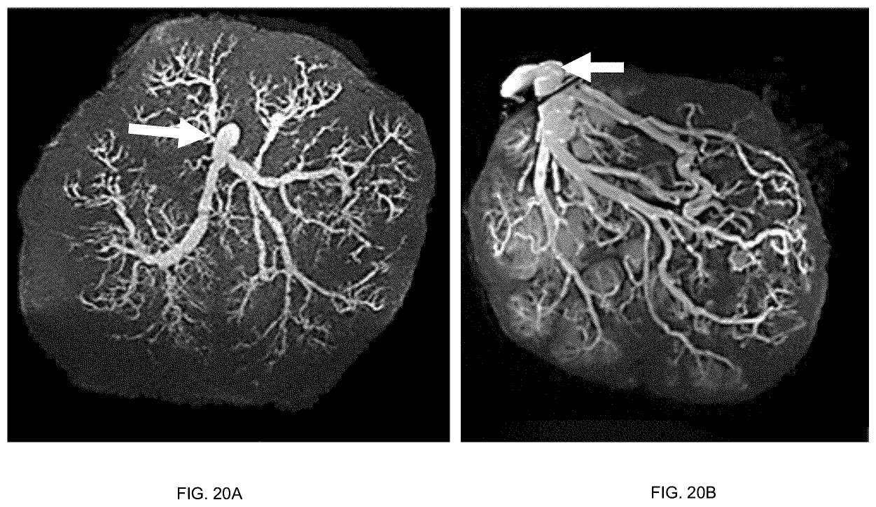

Reference is now made to FIGS. 20A and 20B, which are images depicting T1-weighted MR images of two normal placentas. The insertion point of the umbilical cord is marked by an arrow in both images.

As shown in FIG. 20B, the umbilical cord insertion point is at a marginal point of the placenta. Such a placenta may therefore be referred to as having a marginal umbilical cord insertion point (or a “marginal placenta”, for short). In contrast, and as shown in FIG. 20A, the umbilical cord insertion point is not at a marginal point of the placenta. Such a placenta may therefore be referred to as having a para-central umbilical cord insertion point (or a “paracentral placenta”, for short).

The inventors have experimentally studied relations between placental volumes, umbilical cord insertion points, and a variety of placental features or characteristics and newborn outcomes. Embodiments of the invention may utilize these relations to produce an evaluation 40A or a prediction 40B of a condition of a fetus, as elaborated herein.

For example, paracentral placentas had a significantly larger daughter-to-mother placental diameter ratio, in comparison to marginal placentas. A significant, negative correlation was detected between a distance of the umbilical insertion point from the center of the placenta and the mean daughter-to-mother diameter ratio. In other words, the more marginal a placenta is, the smaller the daughter-to-mother ratio value is.

In another example, strong correlation were detected between the placental volume and the number of blood-vessel bifurcations, and between the number of blood-vessel bifurcations and the number of blood-vessel generations. In other words, the larger the placenta is, the more blood-vessel bifurcations and generations it has.

In another example, strong correlation was found between birth weight and the placental volume.

The term “Pseudo Continuous Arterial Spin Labeling” (PCASL) may be used herein to refer to an MRI technique for measuring tissue perfusion (e.g., blood flow), which uses magnetically labeled arterial blood water protons as an endogenous tracer. By adding a delay between labeling and image acquisition (commonly referred to as post-labeling delay (PLD)), labeled blood is allowed to reach the capillaries where it gives rise to a measurable perfusion signal.

According to some embodiments, system 10 may receive, from scanner 50 a scan of a womb, that includes a plurality of slices 20A. Scan 20 may include, or depict a total fetal body and placental volume. Additionally, or alternatively, volumetric scan 20 may include PCASL information 20′ obtained at least from the placental volume.

For example, PCASL information 20′ may be obtained from scan device 50 by using a multi-delay, 3D inner-volume GRASE free-breathing PCASL scan, with one or more post-labeling delays (PLDs). In some embodiments, the one or more PLDs may include three PLDs, timed at 1000, 1500 and 2000 milliseconds.

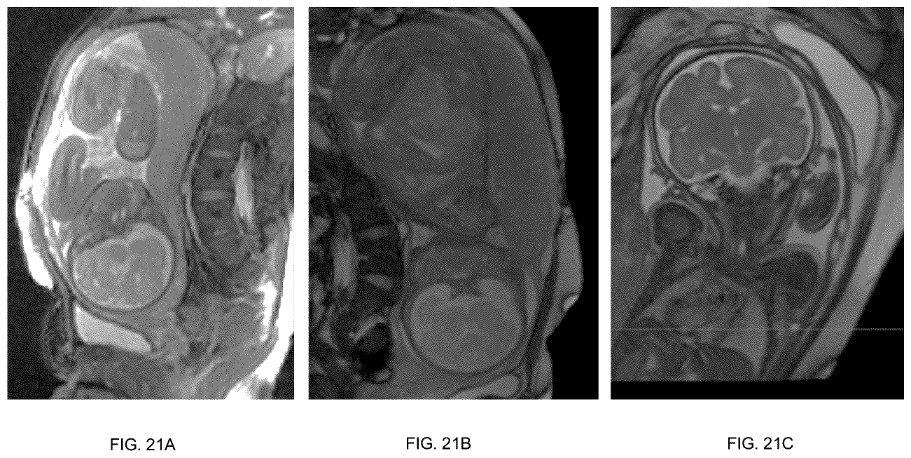

Reference is now made to FIGS. 21A, 21B and 21C, which are images depicting an example of segmentation of a volumetric scan 20, including segmentation of a placenta, a fetus body and fetal brain respectively, as calculated by embodiments of the invention.

As elaborated herein, VOI module 110 may be configured to segment scan 20 to one or more VOIs 110A. The one or more VOIs 110A may include a placental volume VOI 110A, a fetal body VOI 110A, and a fetal brain VOI 110A, as depicted in the example of FIGS. 21A, 21B and 21C, respectively.

Additionally, or alternatively, system 10 may apply at least one ML model 115 on one or more slices 20A of scan 20 (e.g., the sequence of slices) to segment a placental VOI 110A, representing a placenta depicted in the MRI scan 20.

According to some embodiments, and as depicted in FIG. 3, placental features module 300 may include one or more sub-modules, each adapted to extract one or more placental features 300A.

For example, placental features module 300 may include a size module 310, that may be configured to calculate one or more placenta features 300A that are size-related features 310A. For example, size module 310 may calculate a size-related feature 310A that is a placental volume 310A of the segmented placental VOI 110A. In another example, size module 310 may be configured to calculate another size-related feature 310A that is a fetal body volume 310A placental volume, based on the segmented fetal body VOI 110A. In another example, size module 310 may be configured to calculate another size-related feature 310A that is fetal body weight 310A, by multiplying fetal body volume 310A by a predefined specific gravity value. Additional size-related features 310A are also possible.

In another example, placental features module 300 may include an umbilical cord module 340, that may be configured to calculate one or more placenta features 300A that are related to the umbilical cord.

For example, umbilical cord module 340 may be configured to apply an image analysis algorithm, to identify an umbilical cord insertion location in placental vol 110A. Umbilical cord module 340 may subsequently calculate an umbilical cord insertion location score 340A (or umbilical score 340A, for short), which may indicate or quantify the relative distance between the umbilical cord insertion site and the center of mass of the placenta. In other words, the umbilical cord score 340A may representing a marginality of the umbilical cord insertion location in the placenta.

Additionally, umbilical cord module 340 may be configured to calculate umbilical score 340A as a quantification of distance between the umbilical cord insertion site and the center of mass of the placenta, in two planes, resulting in a 3D distance metric.

As elaborated herein, ML model 505 may be trained to receive the one or more placental features 300A (e.g., calculated placental volume 310A and umbilical cord score 340A), and predict one or more respective conditions of the fetus based on the received placental features 300A. For example, ML model 505 may diagnose or evaluate a fetus as currently having a condition such as FGR, placental insufficiency, placental dysfunction, and the like based on the received the one or more placental features 300A.

In another example, ML model 505 may diagnose or evaluate a fetus as currently having a condition such as FGR, placental insufficiency, placental dysfunction, and the like based on the received the one or more placental features 300A (e.g., placental volume 310A and umbilical cord score 340A) and further based on fetal body volume/weight 310A and/or brain volume/weight 310A.

Other combinations of input to ML model 505 consisting of fetal 200A and placental 300A features as elaborated herein may also be possible.

Additionally, or alternatively, ML model 505 may predict a future (e.g., an evolving) fetal condition (FGR, placental insufficiency, etc.) based on concurrent and/or historical placental features 300A.

Additionally, or alternatively, placental features module 300 may include a blood flow module 330, that may be configured to calculate one or more placenta features 300A that are related to blood flow or blood perfusion. Such placenta features 300A may also be referred to herein as vascular metric values 330A.

For example, blood flow module 330 may be, or may include at least on ML model 335. In a training phase, ML model 335 may be trained to receive PCASL information and predict, or calculate one or more vascular metric values 330A based on the received PCASL information 20′. The vascular metric values 330A may include, for example Placental blood flow (PBF) and arterial transit time (ATT). In a subsequent inference phase, ML model 335 may receive a PCASL information 20′ of a target scan 20, and may compute the vascular metric values 330A (e.g., PBF, ATT) based on the received PCASL information and according to the training.

According to some embodiments, ML model 505 may be trained to predict a condition of a fetus further based on the vascular metric values 330A (e.g., PBF, ATT). In a subsequent inference phase, ML model 505 may further receive (e.g., in addition to size-related features 310A, such as placental volume and/or umbilical cord score 340A) vascular metric values 330A (e.g., PBF, ATT) of a target scan 20, and evaluate or predict a condition of the relevant fetus based on the received data.

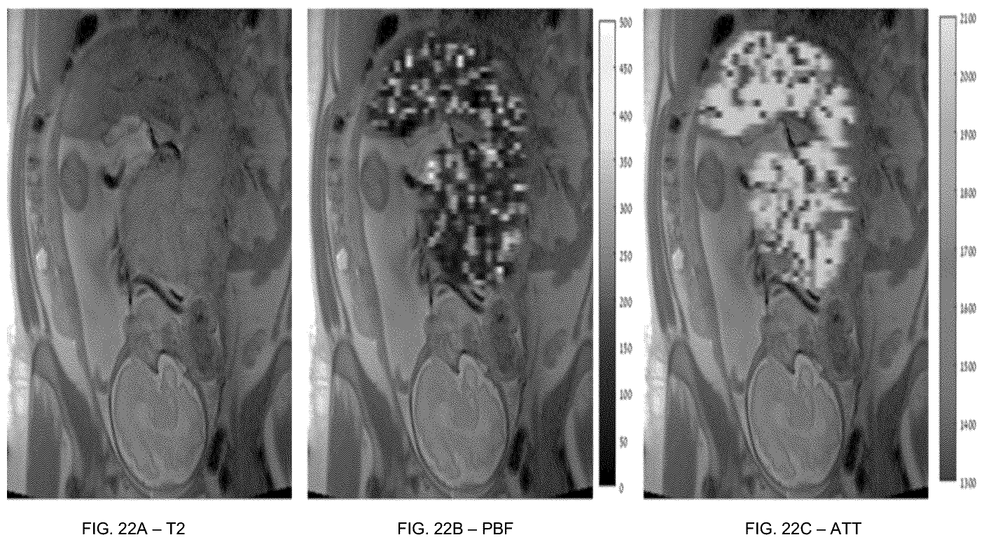

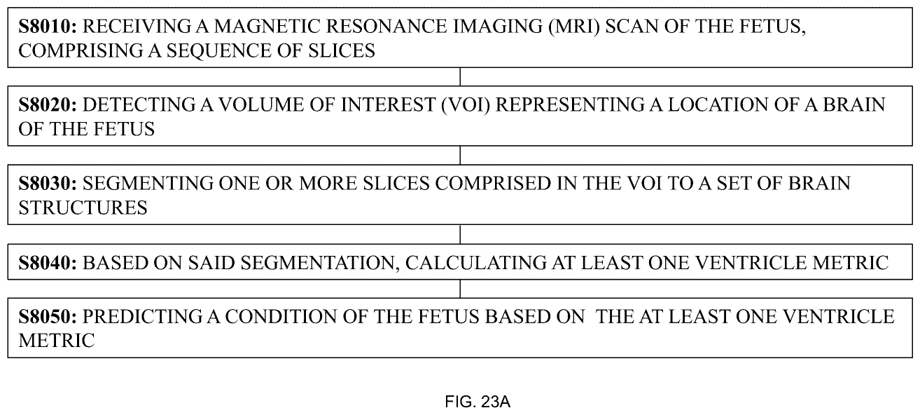

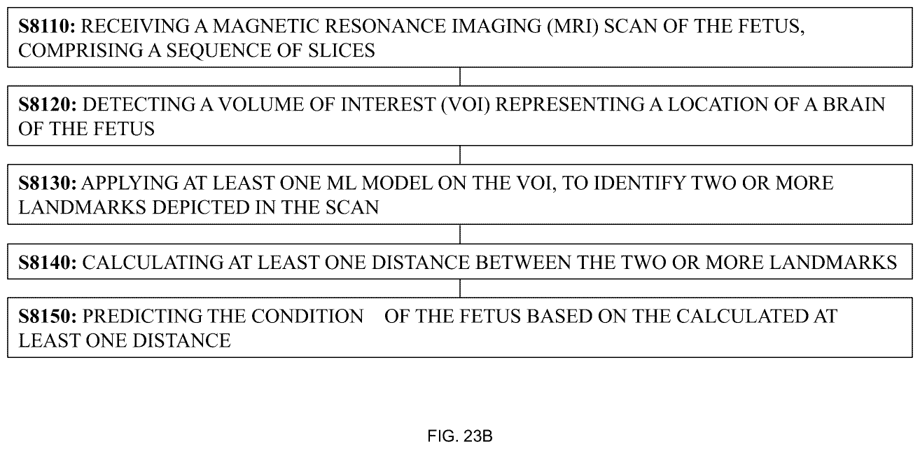

Reference is now made to FIG. 22A, which is an anatomical image depicting a representative T2 scan slice of a womb, accommodating a fetus and a placenta, at a GA of 32 weeks. Reference is further made to FIG. 22B, which is an image depicting values of Placental Blood Flow (PBF), superimposed over the anatomical image of FIG. 22A, as calculated by embodiments of the invention, and to FIG. 22C, which is an image depicting values of Arterial Transit Time (ATT), superimposed over the anatomical image of FIG. 22A, as calculated by embodiments of the invention.

Additionally, or alternatively, placental features module 300 may include an oxygenation module 320, that may be configured to calculate one or more placenta features 300A that are related to blood oxygenation 330A.

For example, oxygenation module 320 may be, or may include at least on ML model 325. In a training phase, ML model 325 may be trained to receive PCASL information and predict, or calculate blood oxygenation value 320A based on the received PCASL information 20′. In a subsequent inference phase, ML model 325 may receive a PCASL information 20′ of a target scan 20, and may compute the blood oxygenation value 320A based on the received PCASL information 20′ and according to the training.

According to some embodiments, ML model 505 may be trained to predict a condition of a fetus further based on the blood oxygenation value 320A. In a subsequent inference phase, ML model 505 may further receive (e.g., in addition to size-related features 310A, such as placental volume, umbilical cord score 340A and/or vascular metric values 330A) blood oxygenation value 320A of a target scan 20, and evaluate or predict a condition of the relevant fetus based on the received data.

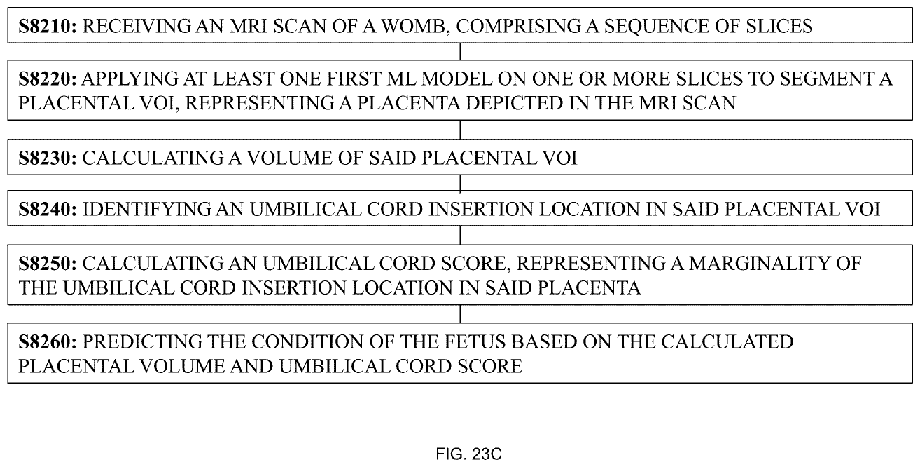

Reference is now made to FIGS. 23A, 23B and 23C, which are flowcharts of methods of predicting a condition of a fetus by at least one processor (such as processor 2 in FIG. 1). As seen in FIG. 23A, according to some embodiments, in step 8010, the at least one processor may receive a magnetic resonance imaging (MRI) scan of the fetus, comprising a sequence of slices. Step 8020 may include detecting, by the at least one processor, a volume of interest (VOI) representing a location of a brain of the fetus. Once a VOI has been detected, step 8030 may include segmenting one or more slices comprised in the VOI to a set of brain structures.

According to some embodiments, the brain structures may be selected from a right hemisphere, a left hemisphere, a right lateral ventricle, and a left lateral ventricle.

According to some embodiments, based on the segmentation, step 8040 may include calculating, by the at least on processor, at least one ventricle metric. The at least one ventricle metric may be selected, according to some embodiments, from: (i) a right lateral ventricle volume, (ii) a left lateral ventricle volume, (iii) an average of volumes of the right and left ventricles, and (iv) asymmetry between volumes of the right and left lateral ventricles.

Step 8050 may include predicting a condition of the fetus based on the at least one ventricle metric. The condition of the fetus may be selected from ventriculomegaly, macrocephaly and microcephaly.

With reference to FIG. 23B, a method of predicting a condition of a fetus by at least one processor according to some embodiments may include, in step 8110 receiving an MRI scan of the fetus, comprising a sequence of slices, and detecting, by the at least one processor, a VOI representing a location of a brain of the fetus depicted in the scan (step 8120).

As may be seen in FIG. 23B, in step 8130, at least one Machine Learning (ML) model may be applied on the VOI, to identify two or more landmarks depicted in the scan.

In step 8140, the at least one processor may calculate at least one distance between the two or more landmarks; and predict (step 8150) the condition of the fetus based on the calculated at least one distance.

According to some embodiments, the condition of the fetus may be one of: hypertelorism, hypotelorism, macrocephaly, microcephaly, ventriculomegaly, and placental dysfunction.

According to some embodiments, the at least one distance is selected from a list of cranial distances consisting of: Cerebral Biparietal Diameter (CBD), Bone Biparietal Diameter (BBD), Trans-Cerebellum Diameter (TCD), front occipital diameter (FOD), Vermian Height (VH), and Lateral Ventricle Width.

According to other embodiments the at least one distance is selected from a list of ocular distances consisting of: Binocular Distance (BOD), Interocular Distance (IOD), Ocular Distance (OD), and Lens Aligned Distance (OD-LA-OD).

FIG. 23C shows another method of predicting a condition of a fetus by at least one processor, according to embodiments of the present invention.

In step 8210, the at least one processor may receive an MRI scan of a womb, comprising a sequence of slices, and may apply, in step 8220, an at least one first ML model on one or more slices of the sequence of slices to segment a placental VOI, representing a placenta depicted in the MRI scan.

In step 8230, the at least on processor may calculate a volume of said placental VOI, and identify in step 8240, an umbilical cord insertion location in said placental VOI.

The at least one processor may, according to some embodiments, calculate an umbilical cord score, representing a marginality of the umbilical cord insertion location in said placenta (step 8250), and in step 8260, may predict the condition of the fetus based on the calculated placental volume and umbilical cord score.

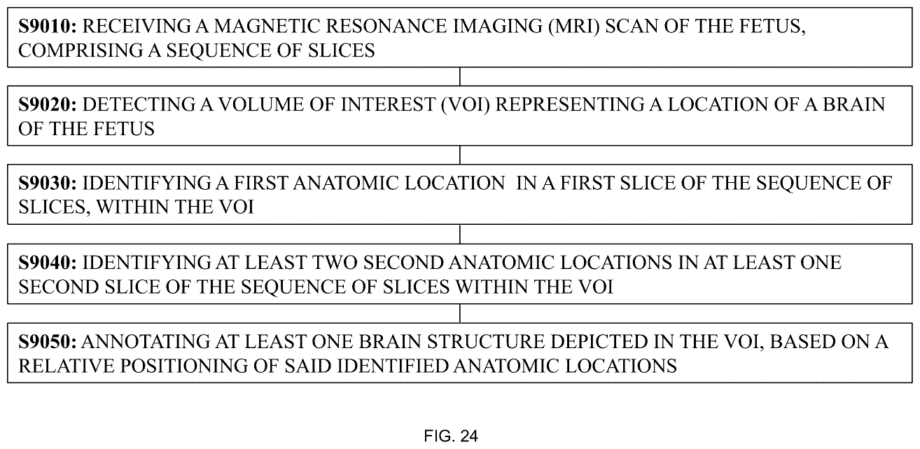

Reference is now made to FIG. 24 which is a flowchart of a method of automatically annotating at least one brain structure in an MRI scan by at least one processor according to embodiments of the present invention. As seen in FIG. 24, in step 9010 an at least one processor, such as processor or controller 2 in FIG. 1, may receive an MRI scan of a fetus, the scan comprising a sequence of slices. In step 9020, the processor may detect a VOI representing a location of a brain of the fetus, depicted in the scan, and identify, in step 9030 a first anatomic location in a first slice of the sequence of slices, within the VOI, and in step 9040, the at least one processor, may identify at least two second anatomic locations in at least one second slice of the sequence of slices within the VOL

In step 9050, the at least one processor may annotate at least one brain structure depicted in the VOI, based on a relative positioning of said identified anatomic locations.

According to some embodiments, the at least one brain structure may be selected from a right hemisphere, a left hemisphere, a right lateral ventricle, and a left lateral ventricle.

Embodiments of the invention thus provide a practical application, based on a fully automated Deep learning (DL) based system, for fetal brain components segmentation, including a separation of right and left hemispheres. Embodiments of the system have been developed and applied using a large clinical cohort. The high performance in cases of structural anomalies such as VM demonstrate the potential applicability of embodiments of this system, to improve diagnosis and assist radiologists in routine clinical practice.

Additionally, embodiments of the invention may provide a practical application for performing automated, DL-based measurements, such as linear distance measurements and volumetric measurements of the fetal brain, as depicted in MRI scans.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of predicting a condition of a fetus by at least one processor, the method comprising:
receiving a magnetic resonance imaging (MRI) scan of the fetus, comprising a sequence of slices;
detecting a volume of interest (VOI) representing a location of a brain of the fetus;
segmenting one or more slices comprised in the VOI to a set of brain structures selected from a right hemisphere, a left hemisphere, a right lateral ventricle, and a left lateral ventricle;
based on said segmentation, calculating at least one ventricle metric, selected from: (i) a right lateral ventricle volume, (ii) a left lateral ventricle volume, (iii) an average of volumes of the right and left ventricles, and (iv) asymmetry between volumes of the right and left lateral ventricles; and
predicting a condition of the fetus based on the at least one ventricle metric.

2. The method of claim 1, wherein the condition of the fetus is selected from ventriculomegaly, macrocephaly and microcephaly.

3. A method of automatically annotating at least one brain structure in an MRI scan by at least one processor, the method comprising:
receiving an MRI scan of a fetus, comprising a sequence of slices;
detecting a VOI representing a location of a brain of the fetus, depicted in the scan;
identifying a first anatomic location in a first slice of the sequence of slices, within the VOI;
identifying at least two second anatomic locations in at least one second slice of the sequence of slices within the VOI; and
annotating at least one brain structure depicted in the VOI, based on a relative positioning of said identified anatomic locations.

4. The method of claim 3, further comprising applying a first ML model on the first slice and on the at least one second slice,
wherein said first ML model is trained to segment each slice of the first slice and the at least one second slice to a plurality of segments,
and wherein identifying the anatomic locations comprises identifying specific segments of the plurality of segments as representing specific brain structures of a predefined set of brain structures.

5. The method of claim 3, further comprising:
determining a direction of the sequence of slices based on said identification of segments; and
annotating the at least one brain structure by applying a label to the one or more segments, wherein said label represents pertinence to a left-side brain structure or a right-side brain structure, based on the determined direction.

6. The method of claim 5, wherein the label is selected from a set of labels consisting of: a right hemisphere, a left hemisphere, a right lateral ventricle, a left lateral ventricle, a cerebellum, a right eye, and a left eye.

7. The method of claim 3, wherein identifying the first anatomic location and the at least two second anatomic locations comprises applying a second ML model on the VOI, wherein said second ML model is trained to identify three or more anatomic locations that are landmarks in the brain, as depicted in slices comprised in the VOI.

8. The method of claim 7, further comprising:
determining a direction of the sequence of slices based on said identification of three or more landmarks; and
annotating the at least one brain structure by applying a label to at least one scan of the sequence of scans,
wherein said label represents a left-right orientation of the depicted brain structure.

9-30. (canceled)

31. A system for predicting a condition of a fetus, the system comprising: a non-transitory memory device, wherein modules of instruction code are stored, and at least one processor associated with the memory device, and configured to execute the modules of instruction code, whereupon execution of said modules of instruction code, the processor is configured to:
receive an MRI scan of the fetus, comprising a sequence of slices;
detect a VOI representing a location of a brain of the fetus depicted in the scan;
apply at least one ML model on the VOI, to identify two or more landmarks depicted in the scan;
calculate at least one distance between the two or more landmarks; and
predict the condition of the fetus based on the calculated at least one distance.

32. The system of claim 31, wherein the at least one distance is selected from a list of cranial distances consisting of: Cerebral Biparietal Diameter (CBD), Bone Biparietal Diameter (BBD), Trans-Cerebellum Diameter (TCD), front occipital diameter (FOD), Vermian Height (VH), and Lateral Ventricle Width.

33. The system of claim 32, wherein the fetal condition is selected from a list consisting of ventriculomegaly, macrocephaly and microcephaly.

34. The system of claim 31, wherein the at least one distance is selected from a list of ocular distances consisting of: Binocular Distance (BOD), Interocular Distance (IOD), Ocular Distance (OD), and Lens Aligned Distance (OD-LA-OD).

35. The system claim 33, wherein the fetal condition is selected from a list consisting of: hypertelorism and hypotelorism.

36. The system of claim 31, wherein the at least one processor is configured to apply at least one ML model by:
applying a first ML model on one or more slices of the sequence of slices, to produce one or more respective slice scores; and
selecting a reference slice from the one or more slices, based on the respective slice scores.

37. The system of claim 36, wherein said first ML model is trained to produce the slice score for each specific slice as a prediction of a probability of selection of the relevant slice by an expert, for the purpose of measuring a specific di stance.

38. The system of claim 36, wherein the at least one processor is further configured to apply a second ML model on a subset of the sequence of slices, comprising the reference slice, to perform multi-class segmentation of the slices to fetal brain structures.

39. The system of claim 38, wherein said second ML model is trained to:
segment each slice to a plurality of segments; and
identify each of the segments as representing a brain structure of a predefined set of brain structures consisting of a right hemisphere, a left hemisphere, and a cerebellum.

40. The system of claim 38, wherein the at least one processor is further configured to:
calculate a midsagittal line in the reference slice, based on said multi-class segmentation;
calculate a brain orientation vector in the reference slice, based on said multi-class segmentation; and
identify the two or more landmarks based on the midsagittal line and the brain orientation vector.

41-44. (canceled)
